SEQUENCE LISTING (16 sequences)

SEQ ID NO: 1 - protein, 77 aa, Artificial Sequence
  A synthetic C3a
SVQLTEKRMDKVGKYPKELRKCCEDGMRENPMRFSCQRRTRFISLGEACKKVFLDCCNYITELRRQHARASHLGLAR

SEQ ID NO: 2 - protein, 5 aa, Artificial Sequence
  A synthetic C3a analog
LPLPR

SEQ ID NO: 3 - protein, 15 aa, Artificial Sequence
  A synthetic EP141
WWGKKYRASKLGLAR

SEQ ID NO: 4 - protein, 5 aa, Artificial Sequence
  A synthetic C3a analog
LPLPR

SEQ ID NO: 5 - protein, 8 aa, Artificial Sequence
  A synthetic C3a analog
PSYLPLPR

SEQ ID NO: 6 - protein, 9 aa, Artificial Sequence
  A synthetic C3a analog
RPSYLPLPR

SEQ ID NO: 7 - protein, 6 aa, Artificial Sequence
  A synthetic C3a analog
FLPLAR

SEQ ID NO: 8 - protein, 6 aa, Artificial Sequence
  A synthetic C3a analog
FIPLAR

SEQ ID NO: 9 - protein, 6 aa, Artificial Sequence
  A synthetic C3a analog
FWTLAR

SEQ ID NO: 10 - protein, 6 aa, Artificial Sequence
  A synthetic C3a analog
FLTLAR

SEQ ID NO: 11 - protein, 6 aa, Artificial Sequence
  A synthetic C3a analog
HLGLAR

SEQ ID NO: 12 - protein, 6 aa, Artificial Sequence
  A synthetic C3a analog
HLALAR

SEQ ID NO: 13 - protein, 9 aa, Artificial Sequence
  A synthetic C3a analog
YSFKDMPXX

SEQ ID NO: 14 - protein, 9 aa, Artificial Sequence
  A synthetic C3a analog
YSFKPMPLX

SEQ ID NO: 15 - protein, 6 aa, Artificial Sequence
  A synthetic C3a analog
FLTXAR

SEQ ID NO: 16 - DNA, 231 bp, Artificial Sequence
  A synthetic C3a analog
tccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccagg

(19) United States
(12) Patent Application Publication
(10) Pub. No.: US 2014/0329761 A1
(43) Pub. Date: Nov. 6, 2014

(54) PREVENTION AND TREATMENT OF ACUTE INFLAMMATORY CONDITIONS

(71) Applicants: Trent Martin Woodruff, Wishart (AU); Stephen Maxwell Taylor, Bellbird Park (AU)

(72) Inventors: Trent Martin Woodruff, Wishart (AU); Stephen Maxwell Taylor, Bellbird Park (AU)

(73) Assignee: The University of Queensland, Queensland (AU)

(21) Appl. No.: 14/128,400

(22) PCT Filed: Jun. 20, 2012
(86) PCT No.: PCT/AU2012/000708

§ 371 Date: Apr. 7, 2014

(30) Foreign Application Priority Data
Jun. 20, 2011 (AU) 2011902394

(51) Int. Cl. A61K 38/10; G01N 33/50; A61K 31/4545; A61K 31/197; A61K 38/08; A61K 31/341

(52) U.S. Cl. CPC A61K 38/10; A61K 38/08; A61K 31/341; A61K 31/4545; A61K 31/197; G01N 33/5023; G01N 2500/10
USPC 514/20.6; 514/471; 514/318; 514/563; 530/326; 530/328; 435/29

(57) ABSTRACT

The present invention relates generally to methods for the prevention and treatment of acute inflammatory conditions in individuals using an agonist of the complement C3a receptor.

### FIELD OF THE INVENTION

This invention relates generally to methods for the prevention and treatment of acute inflammatory conditions in individuals.

### BACKGROUND OF THE INVENTION

Inflammatory conditions can be classified as acute inflammatory conditions or chronic inflammatory conditions. Acute inflammation is the result of the body's initial response to harmful stimuli and is achieved by the increased movement of plasma and leukocytes (especially granulocytes, including neutrophils) from the blood into the injured tissues. A cascade of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, to include immune cells of the mononuclear origin (such as monocytes, lymphocytes and macrophages) and is characterized by simultaneous destruction and healing of the tissue from the inflammatory process.

Those inflammatory conditions that are acute include ischemia or ischemia-reperfusion injuries (e.g., stroke, myocardial infarction, ischemia in other tissues including ischemia in liver, kidney and gut), hemorrhagic shock, conditions associated with trauma (e.g., crush injuries, brain injury, and spinal cord injury), transfusion related acute lung injury (TRALI), acute respiratory distress syndrome (ARDS), trauma or injury associated with surgery (e.g., abdominal aortic aneurysm repair, intestinal surgeries), renal microvasculopathy, acute renal failure, vasculitis, pancreatitis, dermatitis, acute coronary artery syndrome, acute liver injury, acute bacterial infection, and acute tissue injury.

The exact cause of many acute inflammatory conditions is not known, although there is now increasing evidence that the complement pathway is a contributor. Despite this, the precise role of individual components of the pathway remains ambiguous.

### SUMMARY OF THE INVENTION

The present invention arises at least in part from the unexpected discovery that activation of the Complement 3a Receptor (C3aR) decreases granulocyte levels, including, for example levels of mobilized neutrophils, levels of neutrophils infiltrating tissues, and activity levels of neutrophils. The present inventors have determined that this mechanism can be taken advantage of in order to obtain beneficial therapeutic outcomes for those conditions where lower granulocyte (e.g., neutrophil) levels are desired, for example, in order to protect against tissue damage in acute inflammatory conditions.

From the foregoing, it is proposed, in accordance with the present invention, that agonism of C3aR function can assist in preventing or treating an acute inflammatory condition in an individual, as described hereinafter.

Accordingly, in one aspect, the present invention provides methods for preventing or treating an acute inflammatory condition in an individual. These methods generally comprise administering to the individual an effective amount of an agonist of C3aR function. Optionally, the methods include identifying or selecting an individual in need of the prevention or treatment of an acute inflammatory condition. Non-limiting examples of suitable agonists of C3aR function include small organic molecules, nucleic acids, aptamers, peptides, peptidomimetics, polypeptides, proteins, proteoglycans, carbohydrates, sugars, lipids or other organic (carbon containing) or inorganic molecules, including those as further described herein. In some embodiments, the agonist of C3aR function comprises a peptide that comprises, consists essentially of, or consists of an amino acid sequence of or corresponding to the naturally-occurring human C3a sequence (sequence: SVQLTEKRMDKVGKYPKELRKCCEDGMRENPMRFSCQRRTRFISLGEACKKVFL DCCNYITELRRQHARASHLGLAR [SEQ ID NO:1]) or a biologically active fragment thereof, or comprises, consists essentially of, or consists of an amino acid sequence of or corresponding to a variant of the naturally-occurring human C3a sequence or a biologically active fragment thereof. Suitably, the agonist of C3aR function comprises at least about 30, 40, 50, 55, 60, 65, 70, 75, 80, 85, 90, 91, 92, 93, 94, 95, 96, 97, 98, 99 or 100% similarity or identity to the naturally-occurring human C3a sequence, a variant or biologically active fragment thereof. In illustrative embodiments, the agonist of C3aR function comprises, consists or consists essentially of the C-terminal region of the naturally-occurring human C3a sequence, suitably residues 63-77, or a biologically active variant or fragment thereof. In illustrative embodiments, the agonist of C3aR function comprises, consists, or consists essentially of the sequence LPLPR [SEQ ID NO:2] or a biologically active fragment thereof, or a variant of LPLPR [SEQ ID NO:2] or a biologically active fragment thereof. Suitably, the agonist of C3aR function has at least about 30, 40, 50, 55, 60, 65, 70, 75, 80, 85, 90, 91, 92, 93, 94, 95, 96, 97, 98, 99 or 100% similarity or identity to the C-terminal region of the naturally-occurring human C3a sequence, a variant or biologically active fragment thereof.

Another aspect of the present invention contemplates the use of an effective amount of an agent, which is optionally formulated with a pharmaceutically acceptable carrier or diluent, for preventing or treating an acute inflammatory condition in an individual, wherein the agent comprises an agonist of C3aR function as described for example herein. Suitably, the individual has been identified as being in need of the prevention or treatment of an acute inflammatory condition.

In yet another aspect, the present invention resides in the use of an agent in the manufacture of a medicament for preventing or treating an acute inflammatory condition in an individual, wherein the agent comprises an agonist of C3aR function as described, for example, herein. Suitably, the individual has been identified as being in need of the prevention or treatment of an acute inflammatory condition.

According to another aspect, the present invention provides an agonist of C3aR function for preventing or treating an acute inflammatory condition in an individual in need thereof.

In yet another aspect, the invention provides methods for identifying agents that agonize C3aR function for preventing or treating an acute inflammatory condition.

Representative methods comprise contacting a sample of cells expressing C3aR with a test agent. A detected increase in level of activity of C3aR relative to a reference or control level in the absence of the test agent, indicates that the agent agonizes C3aR function and that it is useful for preventing or treating an acute inflammatory condition. Alternatively, or in addition, the methods may comprise determining whether the test agent modulates an acute inflammatory condition parameter (e.g., inflammation, level or activity of neutrophils, neutrophil mobilization, neutrophil infiltration of tissue etc.) wherein a reduction in the parameter (e.g., reduced inflammation, reduced level or activity of neutrophils, reduced neutrophil mobilization, reduced neutrophil infiltration of tissue etc.) indicates that the agent agonizes C3aR function is useful for preventing or treating an acute inflammatory condition. In specific embodiments, the methods comprise determining that the agent agonizes C3aR function and that it reduces an acute inflammatory condition parameter (e.g., inflammation, level or activity of neutrophils, neutrophil mobilization, neutrophil infiltration of tissue etc.), which in representative examples, is reduced neutrophil mobilization.

In accordance with the present invention, the agents identified using the methods broadly described above are useful for preventing or treating an acute inflammatory condition in an individual.

Still another aspect of the present invention provides methods of producing an agent comprising an agonist of C3aR function for preventing or treating an acute inflammatory condition in an individual. These methods generally comprise: testing a test agent as broadly described above; and synthesizing the agent on the basis that it tests positive for agonizing C3aR function. Suitably, the method further comprises derivatizing the agent, and optionally formulating the derivatized agent with a pharmaceutically acceptable carrier or diluent, to improve the efficacy of the agent for treating or preventing an acute inflammatory condition.

### BRIEF DESCRIPTION OF THE DRAWINGS

FIGS. 1-6 show some of the results discussed in Example 1.

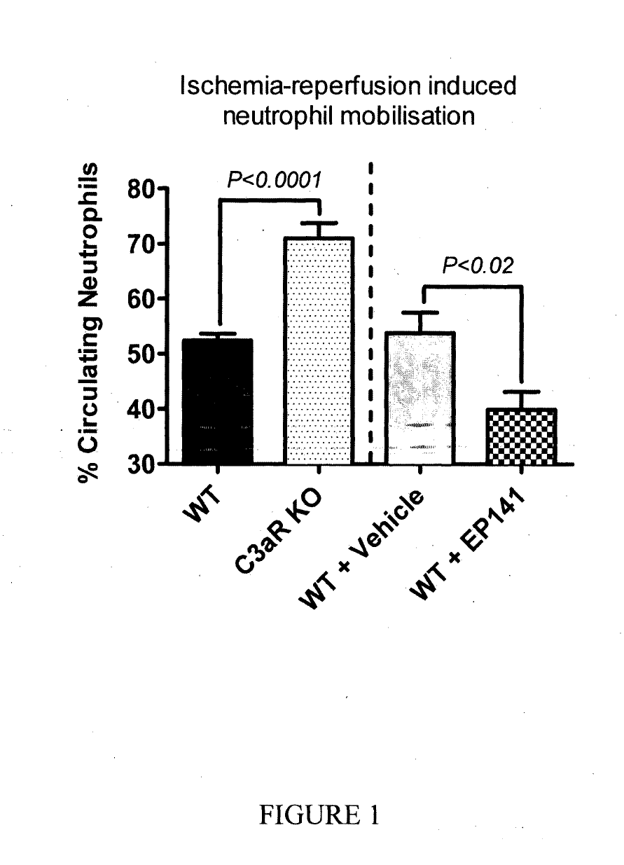

In FIG. 1, the effect of the absence of C3aR, and agonism of C3aR, to neutrophil mobilization in ischemia-reperfusion (I/R) induced intestinal injury is shown, where the left panel shows that the percentage of circulating neutrophils was significantly (P<0.0001) higher in C3aR knockout (KO) mice than wild-type (WT) mice following IR injury. The right panel shows that the percentage of circulating neutrophils was significantly (P<0.02) lower in C3aR agonist (peptide WWGKKYRASKLGLAR [SEQ ID NO:3]; termed EP141) pre-treated WT mice compared with vehicle pre-treated WT mice. The data are expressed as mean±SEM; n=5-9 per group.

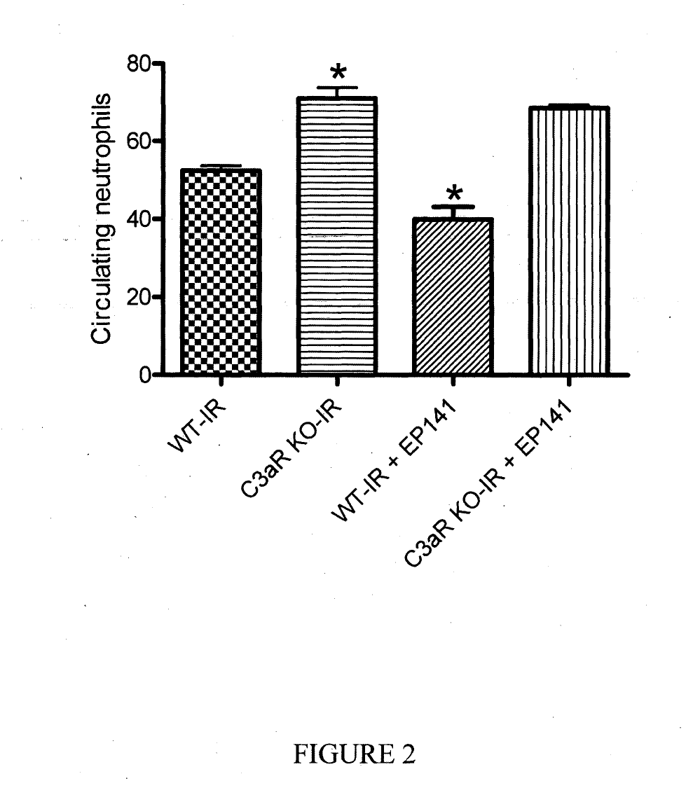

In FIG. 2, the effects of EP141 on circulating neutrophils in both the wild type mice and the C3aR^{-/-} mice in the gut ischemia/reperfusion model can be seen. EP141 was found to be inactive when administered to the C3aR knock-out mice, which demonstrates that EP141 is acting specifically through the C3a receptor in order to reduce neutrophil mobilization.

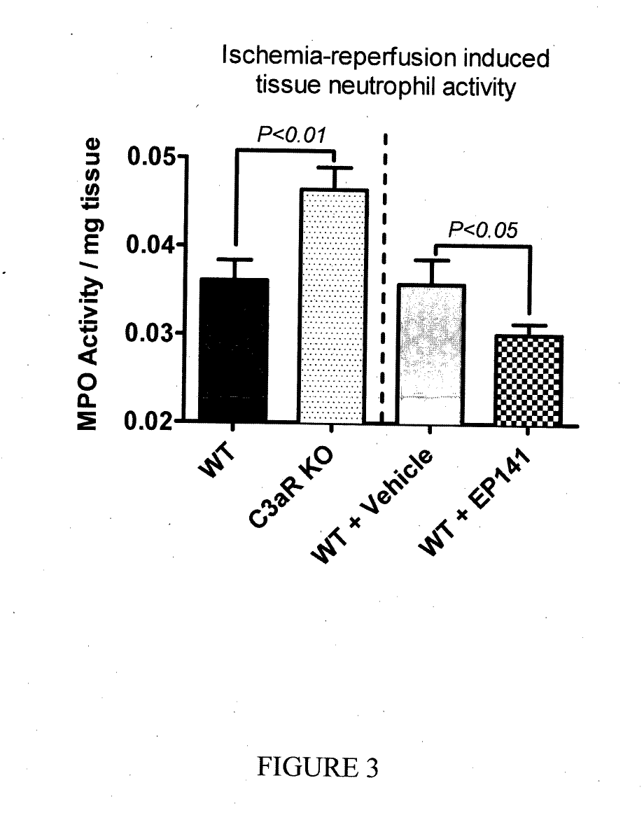

In FIG. 3, the effect of the absence of C3aR, and the agonism of C3aR, on neutrophil activity following I/R induced intestinal injury is indicated by showing myeloperoxidase (MPO) activity of the small intestine. On the left panel, it can be seen that the I/R induced intestinal MPO activity was significantly (P<0.01) greater in C3aR KO mice, compared with wild-type (WT) mice. On the right panel, it can be seen that the I/R induced MPO activity was significantly (P<0.05) lower in C3aR agonist (EP 141) pre-treated WT mice, compared with vehicle pre-treated WT mice. The data are expressed as mean±SEM; n=10-17 per group.

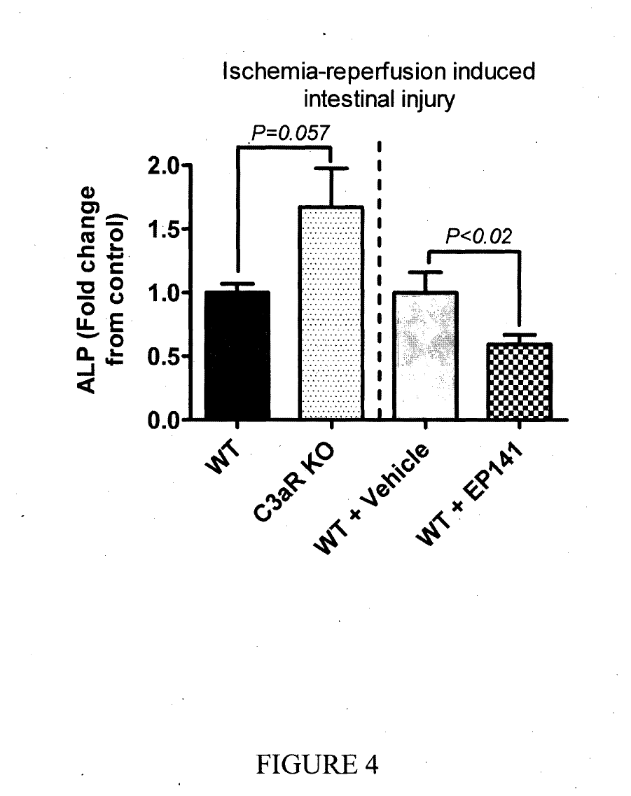

FIG. 4 shows the effect of the absence of C3aR, and agonism of C3aR, on circulating Alkaline Phosphatase (ALP) following I/R induced intestinal injury. As shown in the left panel, ALP levels were higher (P=0.057) in C3aR KO mice compared to wild-type (WT) mice. As shown in the right panel, intestinal ALP levels were significantly (P<0.02) reduced by pre-treatment with the C3aR agonist (EP141) in WT mice, compared with vehicle pre-treated WT mice. Data are expressed as mean±SEM; n=6-17 per group.

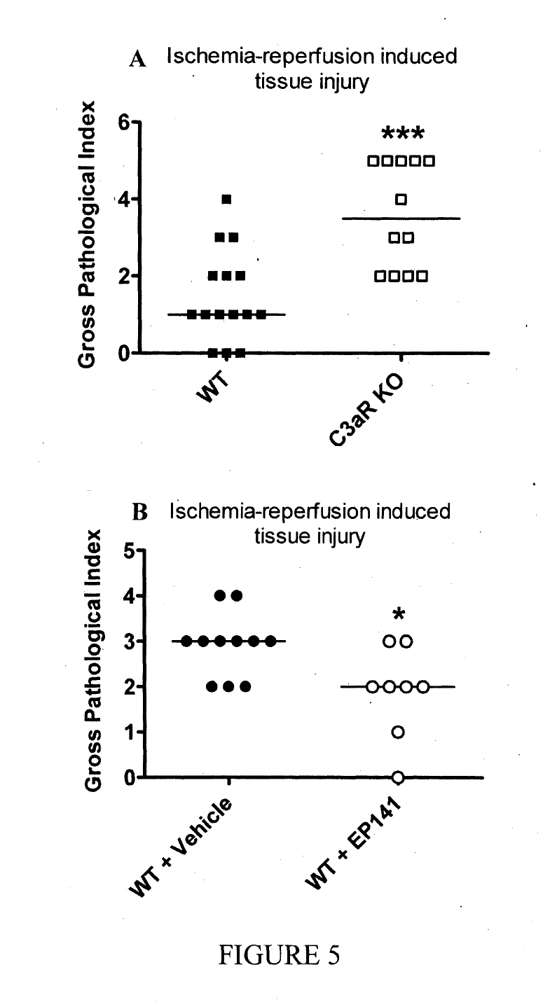

FIG. 5 shows the effect of the absence of C3aR, and agonism of C3aR, to intestinal I/R induced mucosal injury in the mouse small intestine, as histopathological scores of the small intestine. In FIG. 5A, it can be seen that I/R induced intestinal injury was significantly enhanced in C3aR KO mice, compared with wild-type (WT) mice. FIG. 5B shows that I/R induced intestinal injury was significantly reduced in C3aR agonist (EP141) pre-treated WT mice compared with vehicle pre-treated WT mice. The data are expressed as mean±SEM. *p≦0.05 and ***p≦0.001; n=9-15 per group.

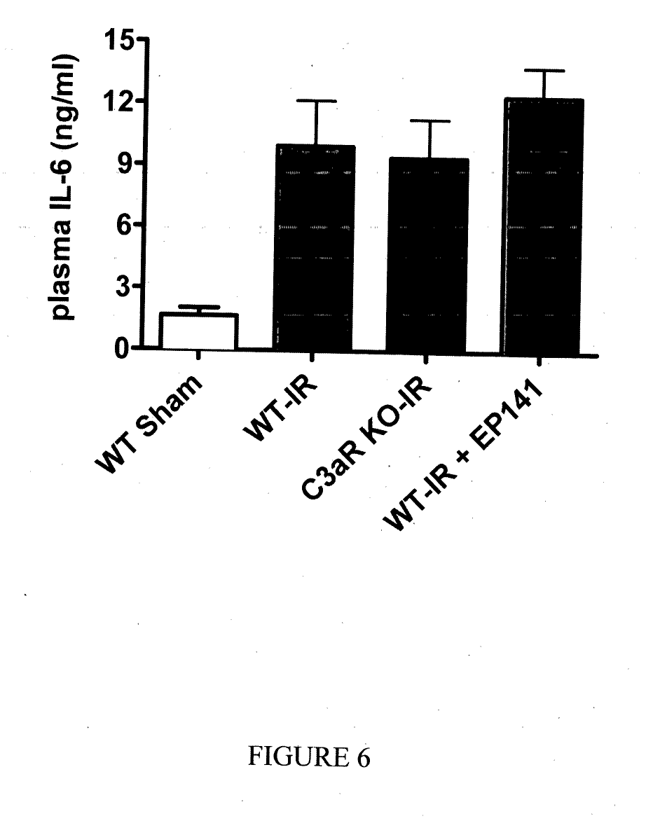

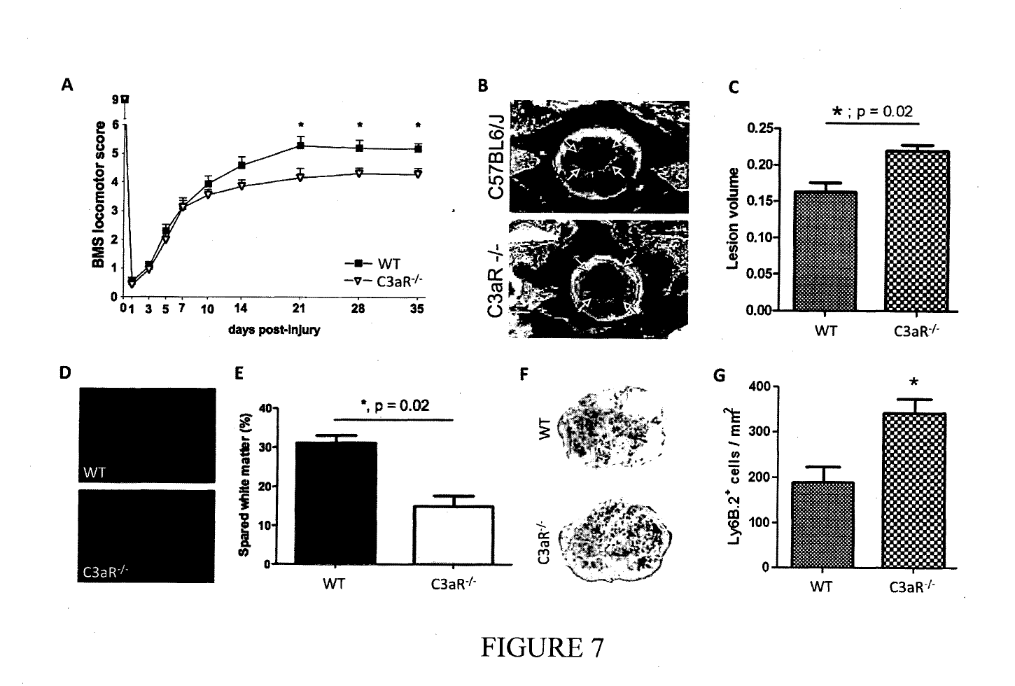

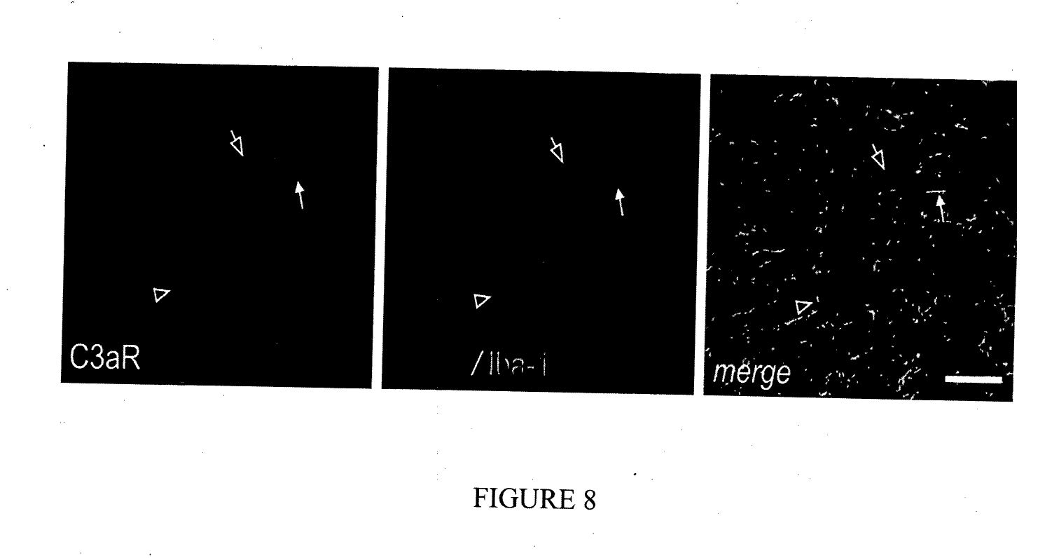

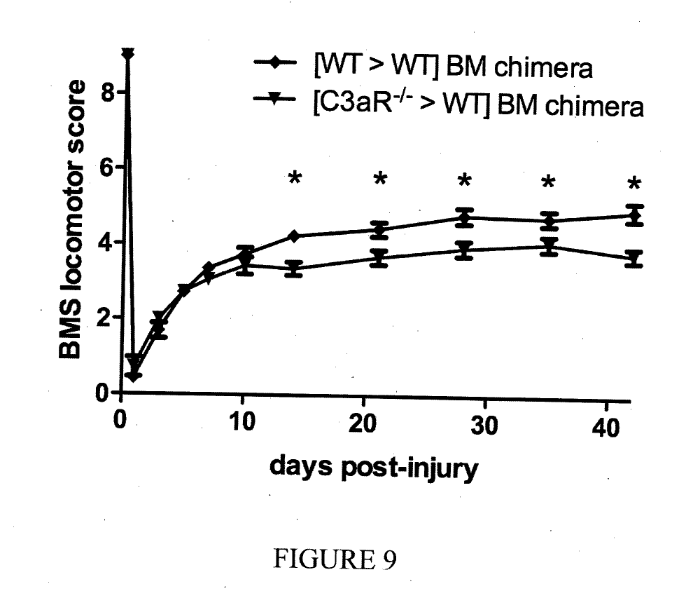

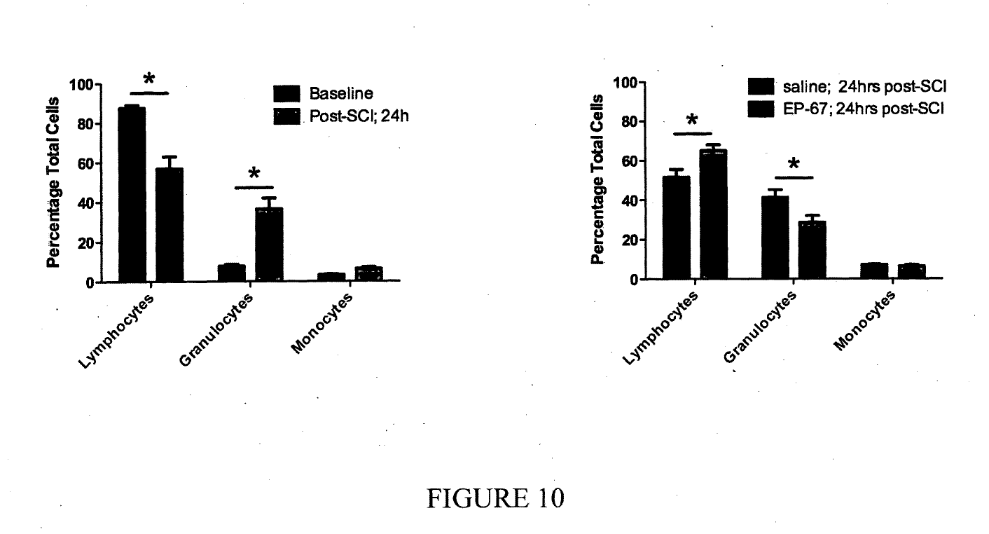

FIG. 6 shows the levels of IL-6 measured in the plasma of the wild type sham mice, wild type ischemia/reperfusion mice, the C3aR^{-/-} ischemia/reperfusion mice and the wild type ischemia/reperfusion mice who had received EP141, 2.5 hours after ischemia (or sham). As can be seen in the graph in FIG. 6, there was no significant change in the IL-6 levels in the C3aR^{-/-} mice and the wild type mice treated with EP 141.

FIGS. 7-10 show some of the results discussed in Example 2.

FIG. 7 shows the first set of results from Example 2, demonstrated that mice deficient in C3aR have a significantly worsened outcome from SCI compared to wild-type mice. See, especially, BMS locator scores as shown in FIG. 7A (n=10-12 per group) where a score ‘9’ represents normal locomotion while ‘0’ is total paralysis. FIGS. 7B and 7C show that the lesion volumes were larger in C3aR^{-/-} mice as determined by MRI, see the arrows in FIG. 7B, and the subsequent quantitative analysis in FIG. 7C. FIGS. 7D and 7E show that the amount of myelin in spared white matter was also significantly decreased, where the light grey in FIG. 7D is the white matter, and FIG. 7E shows the results quantitatively. FIGS. 7F and 7G show that increased neutrophil presence was evident at the lesion site in C3aR^{-/-} mice (n≧6; *, p<0.05).

FIG. 8 shows further results from Example 2 demonstrating that C3aR itself is expressed in the injured spinal cord and its expression is localized to neutrophils, astrocytes and activated microglia/macrophages at the lesion site for at least up to 35 days post-injury. Images shows differential staining for C3aR (left image), GFAP⁺ astrocytes and Iba-1⁺ macrophages (both in middle image), and merged image (right image). The staining shows that C3aR is present on GFAP⁺ astrocytes and Iba-1⁺ macrophages after SCI, and that C3aR⁺ neutrophils do not co-express these markers (arrowhead).

FIG. 9 shows further results from Example 2 demonstrating that expression of C3aR on BM-derived cells is critical for the outcome from SCI. In FIG. 9, the functional phenotype of C3aR deficiency was not rescued in these BM chimeras where C3aR expression was restricted to CNS astrocytes and microglia. It can be seen that the [C3aR^{-/-}>WT] BM chimeric mice have a worse functional outcome from SCI compared to the injured controls. Transplantation of wild-type (WT) bone marrow into irradiated WT recipients was used to exclude the possibility that the chimerization process influenced the outcome from SCI (n=10 per condition; *, p<0.05 [ANOVA & post-hoc]).

FIG. 10 shows more Example 2 results. The left hand graph in FIG. 10 shows the white blood cell distributions (first two bars=lymphocytes; second pair of bars=granulocytes; third pair=monocytes) in uninjured controls (the left bars of each pair—“baseline”) and mice with experimental SCI (the right bars of each pair) at 24 hours post-injury. A dramatic increase in granulocyte numbers in SCI mice as a result of their mobilization in response to injury is shown. The right hand graph in FIG. 10 shows the white blood cell distributions (first two bars=lymphocytes; second pair of bars=granulocytes; third pair=monocytes) 24 hours after SCI, where the left bars of each pair show the results for the saline control and the right bars of each pair show the results for treatment of experimental mice with the C3aR agonist EP67. It can be seen that the EP67 treatment partially counters the granulocyte egress from the BM in response to SCI, resulting in a significant 30% reduction in blood granulocyte numbers. The bars represent mean±SEM; n=4 per experimental condition; asterisk indicates p<0.05.

Some figures contain color representations or entities. Color illustrations are available from the Applicant upon request or from an appropriate Patent Office. A fee may be imposed if obtained from the Patent Office.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

The term “agonist of C3aR function” as used herein refers to any agent which directly or indirectly agonizes or antagonizes a component so as to agonize or otherwise activate or increase the function of the C3aR. In some embodiments, the agonist of C3aR function directly agonizes C3aR function. Suitably, the agonist of C3aR function directly agonizes C3aR function by binding with C3aR and activating the receptor. In some other embodiments, the agonist of C3aR function indirectly agonizes C3aR function. Suitably, the agonist of C3aR function indirectly agonizes C3aR function by directly or indirectly increasing the level of one or more components selected from: a C3 convertase (also known as C4b2a or C3bBb), C3, C3a, and C3aR, or by decreasing the level of one or more components selected from: an extrinsic pathway protease (e.g., serum carboxypeptidase), CD55 and CD46.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity.

“Antigenic or immunogenic activity” refers to the ability of a polypeptide, fragment, variant or derivative according to the invention to produce an antigenic or immunogenic response in an animal, suitably a mammal, to which it is administered, wherein the response includes the production of elements which specifically bind the polypeptide or fragment thereof.

By “biologically active fragment” is meant a fragment of a full-length parent polypeptide which fragment retains an activity of the parent polypeptide. As used herein, the term “biologically active fragment” includes deletion variants and small peptides, for example of at least 6, 7, 8, 9, 10, 11, 12, 13, 14, 15, 16, 17, 18, 19, 20, 21, 22, 23, 24, 25, 26, 27, 28, 29, 30, 35, 40, 45, 50 contiguous amino acid residues, which comprise an activity of the parent polypeptide. Peptides of this type may be obtained through the application of standard recombinant nucleic acid techniques or synthesized using conventional liquid or solid phase synthesis techniques. For example, reference may be made to solution synthesis or solid phase synthesis as described, for example, in Chapter 9 entitled “Peptide Synthesis” by Atherton and Shephard which is included in a publication entitled “Synthetic Vaccines” edited by Nicholson and published by Blackwell Scientific Publications. Alternatively, peptides can be produced by digestion of a polypeptide of the invention with proteinases such as endoLys-C, endoArg-C, endoGlu-C and staphylococcus V8-protease. The digested fragments can be purified by, for example, high performance liquid chromatographic (HPLC) techniques.

The term “biological sample” or “sample” as used herein refers to a sample that may be extracted, untreated, treated, diluted or concentrated from a patient. Suitably, the biological sample is a urine, whole blood, serum or plasma sample.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. Other elements may include, for example, additional amino acid residues at either end of an amino acid sequence, and/or other molecules.

As used herein, “consists essentially of” (and the like) means the inclusion of a stated step or element or group of steps or elements and any additional step/element or steps/elements, provided those additional steps/elements do not interfere with or contribute to the activity or action of the included steps/elements. Thus, when a peptide “consists essentially of” an amino acid sequence corresponding to LPLPR, the amino acid sequence of the peptide may comprise additional amino acid residues (e.g., by as much as 1, 2, 3, 4, 5, 6, 7, 8, 9, 10, 11, 12, 13, 14, 15, 16, 17, 18, 19, 20, 21, 22, 23, 24, 25 or more additional residues) at either end of the amino acid sequence, and/or may be conjugated or otherwise associated with other molecules (e.g., a protecting moiety such as an N-terminal blocking residue (e.g., pyroglutamate)), provided those additional residues or molecules do not substantially modulate the immunogenic properties of the amino acid sequence.

As used herein, the term “consists of” (and the like) means including to, and limited to a stated step or element or group of steps or elements. Thus, when used in context of the sequence of a particular peptide, the phrase “consists of” indicates that the amino acid sequence is a mandatory element, and that no other elements (such as amino acid residues at either end of the amino acid sequence or other molecules) may be present. For example, a peptide consisting of the amino acid sequence corresponding to LPLPR means that the amino acid sequence corresponding to LPLPR is a mandatory element, and that no other elements (such as amino acid residues at either end of the amino acid sequence or other molecules) may be present.

By “corresponds to” or “corresponding to” (and the like) is meant (a) a polynucleotide having a nucleotide sequence that is substantially identical (e.g., one that displays at least 70%, 71, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity to) or complementary to all or a portion of a reference polynucleotide sequence or encoding an amino acid sequence identical to an amino acid sequence in a peptide or protein; or (b) a peptide or polypeptide having an amino acid sequence that displays substantial similarity and/or identity (e.g., one that displays at least 70%, 71, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% identity or similarity) to a sequence of amino acids in a reference peptide or protein.

By “derivative” is meant an agent that has been derived from a basic or parent compound or agent, including by sequence modification, by conjugation or complexing with other chemical moieties or by post-translational modification techniques as would be understood in the art. Methods to produce derivatives of agents, including derivatives of agonists of C3aR function which are also themselves agonists of C3aR function are well known in the art, including methods for derivatizing the agent and methods for testing the activity of the derivative.

By “effective amount”, in the context of preventing or treating an acute inflammatory condition is meant the administration of an amount of active ingredient to an individual in need of such prophylaxis or treatment, either in a single dose or as part of a series, that is effective for prophylaxis or treatment or improvement of an acute inflammatory condition. Non-limiting examples of such improvements in an individual include reducing the symptoms of an acute inflammatory condition, including but not limited to reducing the inflammation in an individual, reducing the size or area of the affected tissue (e.g., inflamed tissue) in an individual, reducing the observable red color in the affected tissue, reducing the size or volume of a lesion in an individual, reducing the level or amount of vascular leakage, reducing the size or volume of edema in the affected tissue, increasing locomotor performance in an individual, increasing mobility in an individual, reducing the level of circulating granulocytes (e.g., neutrophils) in an individual, reducing the number of granulocytes (e.g., neutrophils) in the affected tissue of an individual, reducing infiltration of granulocyte s(e.g., neutrophils) into the affected tissue of an individual, reducing the level of granulocyte (e.g., neutrophil) activity, reducing the amount or activity of an inflammation marker molecule in an individual, including an inflammation marker molecule selected from the group consisting of myeloperoxidase, alkaline phosphatase, and creatine kinase, alanine transaminase, blood urea nitrogen, C-reactive protein, and/or reducing the erythrocyte sedimentation rate (ESR), reducing the level of pain the individual is experiencing, and/or reducing the symptoms of a disease or condition that causes or is otherwise associated with an acute inflammatory condition. The effective amount will vary depending upon the health and physical condition of the individual to be treated, the formulation of the composition being administered, the assessment of the medical situation, and other relevant factors. It is expected that the amount will fall in a relatively broad range that can be determined through routine trials.

The term “expression product” refers to production of mRNA, translation of RNA message into proteins or polypeptides, or processed forms of those proteins or polypeptides.

As used herein, the term “function” refers to a biological, enzymatic, or therapeutic function.

The term “gene” as used herein refers to any and all discrete coding regions of the cell's genome, as well as associated non-coding and regulatory regions. The term “gene” is also intended to mean the open reading frame encoding specific polypeptides, introns, and adjacent 5′ and 3′ non-coding nucleotide sequences involved in the regulation of expression. In this regard, the gene may further comprise control signals such as promoters, enhancers, termination and/or polyadenylation signals that are naturally associated with a given gene, or heterologous control signals. The DNA sequences may be cDNA or genomic DNA or a fragment thereof. The gene may be introduced into an appropriate vector for extrachromosomal maintenance or for integration into the host.

The term sequence “identity” as used herein refers to the extent that sequences are identical on a nucleotide-by-nucleotide basis or an amino acid-by-amino acid basis over a window of comparison. Thus, a “percentage of sequence identity” is calculated by comparing two optimally aligned sequences over the window of comparison, determining the number of positions at which the identical nucleic acid base (e.g., A, T, C, G, U) or the identical amino acid residue (e.g., Ala, Pro, Ser, Thr, Gly, Val, Leu, Ile, Phe, Tyr, Tip, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. For the purposes of the present invention, “sequence identity” may be understood to mean the “match percentage” calculated by the DNASIS computer program (Version 2.5 for Windows; available from Hitachi Software Engineering Co., Ltd., South San Francisco, Calif., USA) using standard defaults as used in the reference manual accompanying the software.

The term sequence “similarity” refer to the percentage number of amino acids that are identical or constitute conservative amino acid substitutions as defined in Table 1 below. Similarity may be determined using sequence comparison programs such as GAP (Deveraux et al., 1984 Nucleic Acids Research 12: 387-395). In this way, sequences of a similar or substantially different length to those cited herein might be compared by insertion of gaps into the alignment, such gaps being determined, for example, by the comparison algorithm used by GAP.

TABLE 1
Exemplary conservative amino acid substitutions

| ORIGINAL | EXEMPLARY |
|---|---|
| RESIDUE | SUBSTITUTIONS |
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile, |
| Phe | Met, Leu, Tyr |
| Ser | Thr |
| Thr | Ser |
| Trp | Tyr |
| Tyr | Trp, Phe |
| Val | Ile, Leu |

Reference herein to “immuno-interactive” includes reference to any interaction, reaction, or other form of association between molecules and in particular where one of the molecules is, or mimics, a component of the immune system.

The terms “individual”, “patient” and “subject” are used interchangeably herein to refer to individuals of human or other animal origin and includes any individual it is desired to examine or treat using the methods of the invention. However, it will be understood that these terms do not imply that symptoms are present. Suitable animals that fall within the scope of the invention include, but are not restricted to, primates, livestock animals (e.g., sheep, cow's, horses, donkeys, pigs), laboratory test animals (e.g., rabbits, Mice, rats, guinea pigs, hamsters), companion animals (e.g., cats, dogs) and captive wild animals (e.g., foxes, deer, dingoes, avians, reptiles).

By “isolated” is meant material that is substantially or essentially free from components that normally accompany it in its native state.

As used herein, the term “mobilization” refers to a process in which cells (e.g., granulocytes such as neutrophils) are released from the bone marrow into the circulation.

By “modulating” is meant increasing or decreasing, either directly or indirectly, the level or functional activity of a target molecule. For example, an agent may indirectly modulate the level/activity by interacting with a molecule other than the target molecule. In this regard, indirect modulation of a gene encoding a target polypeptide includes within its scope modulation of the expression of a first nucleic acid molecule, wherein an expression product of the first nucleic acid molecule modulates the expression of a nucleic acid molecule encoding the target polypeptide.

By “obtained from” is meant that a sample such as, for example, a polynucleotide extract or polypeptide extract is isolated from, or derived from, a particular source of the host. For example, the extract can be obtained from a sample (e.g., tissue or a biological fluid) isolated directly from the host.

The term “oligonucleotide” as used herein refers to a polymer composed of a multiplicity of nucleotide residues (deoxyribonucleotides or ribonucleotides, or related structural variants or synthetic analogues thereof) linked via phosphodiester bonds (or related structural variants or synthetic analogues thereof). Thus, while the term “oligonucleotide” typically refers to a nucleotide polymer in which the nucleotide residues and linkages between them are naturally occurring, it will be understood that the term also includes within its scope various analogues including, but not restricted to, peptide nucleic acids (PNAs), phosphoramidates, phosphorothioates, methyl phosphonates, 2-O-methyl ribonucleic acids, and the like. The exact size of the molecule can vary depending on the particular application. An oligonucleotide is typically rather short in length, generally from about 10 to 30 nucleotide residues, but the term can refer to molecules of any length, although the term “polynucleotide” or “nucleic acid” is typically used for large oligonucleotides.

By “operably linked” is meant that transcriptional and translational regulatory polynucleotides are positioned relative to a polypeptide-encoding polynucleotide in such a manner that the polynucleotide is transcribed and the polypeptide is translated.

By “pharmaceutically acceptable carrier or diluent” is meant a solid or liquid filler, diluent or encapsulating substance that can be safely used in topical or systemic administration to a mammal.

The term “polynucleotide” or “nucleic acid” as used herein designates mRNA, RNA, cRNA, cDNA or DNA. The term typically refers to oligonucleotides greater than 30 nucleotide residues in length.

The terms “polynucleotide variant” and “variant” refer to polynucleotides displaying substantial sequence identity with a reference polynucleotide sequence or polynucleotides that hybridize with a reference sequence under stringent conditions as known in the art (see for example Sambrook et al., Molecular Cloning. A Laboratory Manual”, Cold Spring Harbor Press, 1989). These terms also encompass polynucleotides in which one or more nucleotides have been added or deleted, or replaced with different nucleotides. In this regard, it is well understood in the art that certain alterations inclusive of mutations, additions, deletions and substitutions can be made to a reference polynucleotide whereby the altered polynucleotide retains a biological function or activity of the reference polynucleotide. The terms “polynucleotide variant” and “variant” also include naturally-occurring allelic variants.

“Polypeptide”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally occurring amino acid, such as a chemical analogue of a corresponding naturally occurring amino acid, as well as to naturally occurring amino acid polymers.

The term “polypeptide variant” refers to polypeptides in which one or more amino acids have been replaced by different amino acids. It is well understood in the art that some amino acids may be changed to others with broadly similar properties without changing the nature of the activity of the polypeptide (conservative substitutions). These terms also encompass polypeptides in which one or more amino acids have been added or deleted, or replaced with different amino acids.

The phrase “preventing or treating an acute inflammatory condition”, and similar, as used herein, includes the prophylaxis or treatment of an acute inflammatory condition, the prophylaxis or treatment of a symptom of an acute inflammatory condition, or the prophylaxis or treatment of a symptom of a disease or condition that causes or is associated with an acute inflammatory condition.

“Probe” refers to a molecule that binds to a specific sequence or sub-sequence or other moiety of another molecule. Unless otherwise indicated, the term “probe” typically refers to a polynucleotide probe that binds to another polynucleotide, often called the “target polynucleotide”, through complementary base pairing. Probes can bind target polynucleotides lacking complete sequence complementarity with the probe, depending on the stringency of the hybridization conditions. Probes can be labeled directly or indirectly.

The term “recombinant polynucleotide” as used herein refers to a polynucleotide formed in vitro by the manipulation of a polynucleotide into a form not normally found in nature. For example, the recombinant polynucleotide can be in the form of an expression vector. Generally, such expression vectors include transcriptional and translational regulatory polynucleotide operably linked to the polynucleotide.

By “recombinant polypeptide” is meant a polypeptide made using recombinant techniques, i.e., through the expression of a recombinant or synthetic polynucleotide.

The term “reference or control level” as used herein refers to any suitable reference or control level, including, but not limited to, a normal healthy individual, an individual without an acute inflammatory condition, a level measured in the same individual at a different time, and a level measured in a biological sample taken from a different tissue in the same individual or control.

By “reporter molecule” as used in the present specification is meant a molecule that, by its chemical nature, provides an analytically identifiable signal that allows the detection of a complex comprising an antigen-binding molecule and its target antigen. The term “reporter molecule” also extends to use of cell agglutination or inhibition of agglutination such as red blood cells on latex beads, and the like.

The term “selective agonist of C3aR function” refers to agonists of C3a receptor (C3aR) function as herein defined that agonize or otherwise activate or increase the function of the C3aR without displaying substantial agonism of one or more other receptors. Accordingly, a selective agonist of C3aR function exhibits C3aR selectivity of greater than about 2-fold, 5-fold, 10-fold, 20-fold, 50-fold or greater than about 100-fold with respect to agonism towards one or more other receptors (i.e., C5aR). In some embodiments, selective agonists of C3aR function display at least 2-fold, 5-fold, 10-fold, at least 20-fold, at least 50-fold, at least 100-fold, at least 500-fold, at least 1000-fold greater agonism towards C3aR than towards C5aR. In other embodiments, selective agonists of C3aR function display at least 10-fold, at least 20-fold, at least 50-fold, at least 100-fold, at least 500-fold, at least 1000-fold greater agonism towards both C3aR and C5aR than of another receptor. In other embodiments, selective agonists of C3aR function display at least 10-fold, at least 20-fold, at least 50-fold, at least 100-fold, at least 500-fold, at least 1000-fold greater agonism towards both C3aR and C5aR than of another complement receptor.

As used herein a “small molecule” refers to a composition that has a molecular weight of less than 3 kilodaltons (kDa), and typically less than 1.5 kilodaltons, and more preferably less than about 1 kilodalton. Small molecules may be nucleic acids, peptides, polypeptides, peptidomimetics, carbohydrates, lipids or other organic (carbon-containing) or inorganic molecules. As those skilled in the art will appreciate, based on the present description, extensive libraries of chemical and/or biological mixtures, often fungal, bacterial, or algal extracts, may be screened with any of the assays of the invention to identify compounds that modulate a bioactivity. A “small organic molecule” is an organic compound (or organic compound complexed with an inorganic compound (e.g., metal)) that has a molecular weight of less than 3 kilodaltons, less than 1.5 kilodaltons, or even less than about 1 kDa.

With reference to the term “stereoisomers” as used herein, it will also be recognized that the compounds described herein may possess asymmetric centers and are therefore capable of existing in more than one stereoisomeric form. The invention thus also relates to compounds in substantially pure isomeric form at one or more asymmetric centers e.g., greater than about 90% ee, such as about 95% or 97% ee or greater than 99% ee, as well as mixtures, including racemic mixtures, thereof. Such isomers may be naturally occurring or may be prepared by asymmetric synthesis, for example using chiral intermediates, or by chiral resolution.

By “vector” is meant a polynucleotide molecule, suitably a DNA molecule derived, for example, from a plasmid, bacteriophage, yeast or virus, into which a polynucleotide can be inserted or cloned. A vector may contain one or more unique restriction sites and can be capable of autonomous replication in a defined host Cell including a target cell or tissue or a progenitor cell or tissue thereof, or be integrable with the genome of the defined host such that the cloned sequence is reproducible. Accordingly, the vector can be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector can contain any means for assuring self-replication. Alternatively, the vector can be one which, when introduced into the host cell, is integrated into the genome and replicated together with the chromosome(s) into which it has been integrated. A vector system can comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. In the present case, the vector is suitably a viral or viral-derived vector, which is operably functional in animal and suitably mammalian cells. Such vector may be derived from a poxvirus, an adenovirus or yeast. The vector can also include a selection marker such as an antibiotic resistance gene that can be used for selection of suitable transformants. Examples of such resistance genes are known to those of skill in the art and include the nptII gene that confers resistance to the antibiotics kanamycin and G418 (Geneticin®) and the hph gene which confers resistance to the antibiotic hygromycin B.

The terms “wild-type” and “normal” are used interchangeably to refer to the phenotype that is characteristic of most of the members of the species occurring naturally and contrast for example with the phenotype of a mutant.

As used herein, underscoring or italicizing the name of a gene shall indicate the gene, in contrast to its protein product, which is indicated by the name of the gene in the absence of any underscoring or italicizing. For example, “C3aR” shall mean the gene encoding the complement factor 3a receptor, whereas “C3aR” shall indicate the product generated from transcription and translation and alternative splicing of the “C3aR” gene (i.e., the complement factor 3a receptor).

### 2. Prevention and Treatment Methods

The present invention arises at least in part from the unexpected discovery that activation of the C3a receptor (C3aR) decreases granulocyte levels, including for example levels of mobilized neutrophils, levels of neutrophils infiltrating tissues, and activity levels of neutrophils. These effects were observed even in the presence of endogenous C3a after an acute inflammatory state was induced. It was also observed that these effects appear to be independent of any change to cytokine levels.

The present inventors have determined that this mechanism can be taken advantage of in order to obtain beneficial therapeutic outcomes for those conditions where lower granulocyte (e.g., neutrophil) levels are desired, for example, in order to protect against tissue damage in an acute inflammatory conditions. In acute inflammatory states, it is desirable to reduce granulocyte (e.g., neutrophil) levels because granulocytes (e.g., neutrophils) can have a direct harmful effect on the body, for example by causing tissue damage. This can be contrasted with chronic inflammatory states (for example, sepsis) where an increased level of granulocytes (e.g., neutrophils) or an increased level of granulocyte (e.g., neutrophil) activity is desirable because it can help to treat the body (for example, clear bacteria in an individual suffering from sepsis), or to promote tissue healing.

Accordingly, the present inventors propose that agonism of C3aR function can assist in preventing or treating acute inflammatory conditions, namely those conditions where the increased mobilization, infiltration and/or activity of granulocytes (e.g., neutrophils) plays a role in the pathogenesis of the conditions and where it is thus desirable to decrease the mobilization, infiltration and/or activity of these granulocytes neutrophils).

In one aspect, the present invention thus provides agonists of C3aR function for preventing or treating an acute inflammatory condition in an individual in need thereof. In another aspect, the present invention provides methods for preventing or treating an acute inflammatory condition in an individual comprising administering to the individual an effective amount of an agonist of C3aR function. Another aspect of the present invention contemplates the use of an effective amount of an agent, which is optionally formulated with a pharmaceutically acceptable carrier or diluent, for preventing or treating an acute inflammatory condition in an individual, wherein the agent comprises an agonist of C3aR function as described for example herein. Also contemplated by the present invention is the use of an agent in the manufacture of a medicament for preventing or treating an acute inflammatory condition in an individual, wherein the agent comprises an agonist of C3aR function as described, for example, herein.

Optionally, the aspects of the present invention include a first step of identifying an individual in need of the prevention or treatment of an acute inflammatory condition.

The individual may or may not have been diagnosed with an acute inflammatory condition or a disease or condition that is associated with or causes an acute inflammatory condition. In some embodiments, the acute inflammatory condition is idiopathic. In some other embodiments, the acute inflammatory condition is caused by or associated with a disease or condition in the individual (whether diagnosed or undiagnosed). Many acute inflammatory conditions are well known in the art, including ischemia or ischemia-reperfusion injuries (e.g., stroke, myocardial infarction, ischemia in other tissues including ischemia in liver, kidney and gut), hemorrhagic shock, conditions associated with trauma (e.g., crush injuries, brain injury, and spinal cord injury), transfusion related acute lung injury (TRALI), acute respiratory distress syndrome (ARDS), trauma or injury associated with surgery (e.g., abdominal aortic aneurysm repair, intestinal surgeries), renal microvasculopathy, acute renal failure, vasculitis, pancreatitis, dermatitis, acute coronary artery syndrome, acute liver injury, acute bacterial infection, and acute tissue injury. In some embodiments, the individual is not suffering from a chronic inflammatory condition. In some embodiments, the individual is not an individual suffering from a neurodegenerative disease or a neurological injury.

Neutrophils are known to be associated with inflammation. Symptoms of inflammation include an increased level of circulating neutrophils, and/or infiltration of neutrophils into the affected tissue (i.e., a higher number of neutrophils in the affected tissue), and/or an increased level of neutrophil activity. In chronic inflammation, neutrophils may play a role in tissue repair. However, in acute inflammatory conditions, these effects are undesirable because they have the potential to cause direct local tissue damage in normal tissue (see, for example, Segel et al. 2011).

Neutrophils release many tissue destructive enzymes, such as myeloperoxidase (MPO) when activated, and thus levels of MPO or MPO activity levels can be used to determine whether neutrophil-based inflammation exists, and also whether prevention or treatment of an acute inflammatory condition is occurring. MPO levels or MPO activity can be measured using any suitable method known in the art, including those disclosed in Woodruff et al. 2005.

There are also symptoms of an acute inflammatory condition that are idiosyncratic to the affected tissue. For example, acute inflammation in intestinal tissue results in an increase in circulating alkaline phosphatase (ALP), while acute inflammation in cardiac tissue results in an increase in creatine kinase (CK or CK-MB). Liver damage results in an increase in plasma alanine transaminase (ALT), and kidney damage results in an increase in plasma blood urea nitrogen (BUN). C-reactive protein (CRP) is also an acute phase inflammatory protein whose levels rise in response to inflammation. Suitable methods for measuring ALP, CK or CK-MB, ALT, BUN, and CRP levels or activity are known in the art, and include those described in Woodruff et al. 2004.

Accordingly, the molecules MPO, ALP, CK or CK-MB, ALT, BUN and CRP, amongst others, are considered “inflammation marker molecules” as a change in their levels can indicate a change in the acute inflammatory state of an individual.

It is proposed, therefore, that agonists of C3aR function will be useful inter alia for the prevention or treatment of an acute inflammatory condition in an individual, including reducing the symptoms of an acute inflammatory condition, including but not limited to reducing the inflammation in an individual, reducing the size or area of the affected tissue (e.g., inflamed tissue) in an individual, reducing the observable red color in the affected tissue, reducing the size or volume of a lesion in an individual, reducing the level or amount of vascular leakage, reducing the size or volume of edema in the affected tissue, increasing locomotor performance in an individual, increasing mobility in an individual, reducing the level of circulating granulocytes (e.g., neutrophils) in an individual, reducing the number of granulocytes (e.g., neutrophils) in the affected tissue of an individual, reducing infiltration of granulocytes (e.g., neutrophils) into the affected tissue of an individual, reducing the level of granulocyte (e.g., neutrophil) activity, reducing the amount or activity of an inflammation marker molecule in an individual, including an inflammation marker molecule selected from the group consisting of myeloperoxidase, alkaline phosphatase, and creatine kinase, alanine transaminase, blood urea nitrogen, C-reactive protein, and/or reducing the erythrocyte sedimentation rate (ESR), reducing the level of pain the individual is experiencing, and/or reducing the symptoms of a disease or condition that causes or is otherwise associated with an acute inflammatory condition.

### 3. Agents that Agonize C3aR Function

Non-limiting examples of suitable agonists of C3aR function include small organic molecules, nucleic acids, aptamers, peptides, peptidomimetics, polypeptides, proteins, proteoglycans, carbohydrates, sugars, lipids or other organic (carbon containing) or inorganic molecules, including those as further described herein.

In some embodiments, the agonist of C3aR function modulates the expression of a gene or the level or functional activity of an expression product of the gene, wherein the gene encodes a component of the complement pathway associated with C3aR function. Representative genes encoding a component of the complement pathway associated with C3aR function include C3 and C3aR. Representative expression products of genes encoding components of the complement pathway associated with C3aR function include a C3 convertase, C3, C3a, and C3aR.

In other embodiments, the agonist of C3aR function modulates the expression of a gene or the level or functional activity of an expression product of the gene, wherein the gene encodes an expression product which modulates directly or indirectly the expression of a gene encoding a component of the complement pathway associated with C3aR function.

In other embodiments, the agonist of C3aR function modulates the expression of a gene or the level or functional activity of an expression product of the gene, wherein the gene encodes an expression product which modulates directly or indirectly the expression product of a gene encoding a component of the complement pathway associated with C3aR function.

The agonist of C3aR function suitably modulates the expression of the gene directly, or modulates an upstream regulator of the expression of the gene, or directly or indirectly modulates the level or functional activity of an expression product of such genes.

In some embodiments, the agonist of C3aR function increases the expression of a gene (e.g., C3, C3aR) or the level or functional activity of an expression product of that gene (e.g., a C3 convertase, C3, C3a, C3aR). In other embodiments, the agonist of C3aR function decreases the expression of a gene or the level or functional activity of an expression product of that gene (e.g., an inhibitor of C3a production such as CD55 or CD46; or an enzyme responsible for C3a inactivation or degradation including an extrinsic pathway protease such as serum carboxypeptidase).

Suitably, the agonist of C3aR function increases or reduces the expression of the gene or the level of functional activity of an expression product of that gene by at least 10%, 20%, 30%, 40%, 50%, 60%, 70%, 80%, 90% relative to the expression, level or functional activity in the absence of the agonist of C3aR function.

Suitable agents for reducing or abrogating gene expression include, but are not restricted to: oligoribonucleotide sequences, including anti-sense RNA and DNA molecules and ribozymes, that function to inhibit the translation of mRNA. Anti-sense RNA and DNA molecules act to directly block the translation of mRNA by binding to targeted mRNA and preventing protein translation. In regard to antisense DNA, oligodeoxyribonucleotides derived from the translation initiation site, e.g., between −10 and +10 regions may be used. Ribozymes are enzymatic RNA molecules capable of catalyzing the specific cleavage of RNA. Alternatively, RNA molecules that mediate RNA interference (RNAi) of a target gene or gene transcript can be used to reduce or abrogate gene expression.

Suitable agents for modulating the level or functional activity of an expression product of a gene include, but are not restricted to small organic molecules, nucleic acids, aptamers, peptides, polypeptides, proteins, proteoglycans, peptidomimetics, carbohydrates, sugars, lipids or other organic (carbon containing) or inorganic molecules, as further described herein.

Suitably, the C3aR is the subject of the targeting. For example, agonism or other activation of receptor signaling is achieved through increase in receptor expression or receptor mutation (in particular, but not exclusively, of phosphorylation sites). Such strategies include antigen-binding molecules (e.g., antibodies) to the receptors and small molecule agonists of binding. Pharmacological strategies to agonize receptor phosphorylation can also be effective.

In some embodiments, the agonist of C3aR function may agonize both the function of the C3aR and at least one other receptor. In other embodiments, the agonist of C3aR function is a selective C3aR agonist, and/or a specific agonist of C3aR function.

In some embodiments, the agonist of C3aR function comprises a peptide that comprises, consists essentially of, or consists of an amino acid sequence of or corresponding to the naturally-occurring human C3a sequence (sequence: SVQLTEKRMDKVGKYPKELRKCCEDGMRENPMRFSCQRRTRFISLGEACKKVFL DCCNYITELRRQHARASHLGLAR [SEQ ID NO:1]) or a biologically active fragment thereof, or comprises, consists essentially of, or consists of an amino acid sequence of or corresponding to a variant of the naturally-occurring human C3a sequence or a biologically active fragment thereof. Suitably, the agonist of C3aR function comprises at least about 30, 40, 50, 55, 60, 65, 70, 75, 80, 85, 90, 91, 92, 93, 94, 95, 96, 97, 98, 99 or 100% similarity or identity to the naturally-occurring human C3a sequence, a variant or biologically active fragment thereof. In illustrative embodiments, the agonist of C3aR function comprises, consists or consists essentially of the C-terminal region of the naturally-occurring human C3a sequence, suitably residues 63-77, or a biologically active variant or fragment thereof. In illustrative embodiments, the agonist of C3aR function comprises, consists, or consists essentially of the sequence LPLPR [SEQ ID NO:2] or a biologically active fragment thereof, or a variant of LPLPR [SEQ ID NO:2] or a biologically active fragment thereof. Suitably, the agonist of C3aR function at least about 30, 40, 50, 55, 60, 65, 70, 75, 80, 85, 90, 91, 92, 93, 94, 95, 96, 97, 98, 99 or 100% similarity or identity to the C-terminal region of the naturally-occurring human C3a sequence, a variant or biologically active fragment thereof.

In certain embodiments, the amino acid sequence of the peptide may differ from the C3a sequence (or fragment thereof) by at least one amino acid substitution, addition, and/or deletion. Substituted amino acids may include conservative amino acid substitutions. Non-conservative substitutions may be tolerated, depending on the location of the substituted residues in the peptide, and other factors known to those skilled in the art. Exemplary conservative substitutions are shown in Table 1, above. Alternatively or in addition, substituted amino acids or added amino acids can be any non-naturally occurring amino acids or derivatives thereof. Non-naturally occurring amino acids include chemical analogues of a corresponding naturally occurring amino acid. Examples of unnatural amino acids and derivatives include, but are not limited to, 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, ornithine, sarcosine, 2-thienyl alanine and/or D-isomers of amino acids.

In some embodiments, the peptide that comprises, consists essentially of, or consists of an amino acid sequence of or corresponding to the naturally-occurring human C3a sequence, or a biologically active fragment thereof, or a variant of the naturally-occurring human C3a sequence, or a biologically active fragment thereof, is or is a derivative of a homolog or isoform of the naturally-occurring human C3a sequence, or displays at least about 30, 40, 50, 55, 60, 65, 70, 75, 80, 85, 90, 91, 92, 93, 94, 95, 96, 97, 98, or 99% similarity or identity to a homolog or isoform of the naturally-occurring C3a sequence. A “homolog” is a molecule from a different species and which is related by descent from a common ancestral DNA sequence. The term “homolog” may apply to the relationship between genes separated by the event of speciation or to the relationship between genes separated by the event of genetic duplication. Human, companion animals and livestock C3a homologs are known. An “isoform” is a peptide that has the same function as another peptide but which is encoded by a different polynucleotide and may have small differences in its sequence.

The peptide may be a biologically active fragment of an amino acid sequence corresponding to the naturally-occurring C3a sequence or a variant thereof. Reference herein to a “fragment” means a molecule which contains at least about four contiguous amino acids. The biologically active fragment may have the C3aR agonist activity associated with the full-length native C3a sequence and/or may have an altered activity. An “altered activity” includes an enhanced activity or loss of a detrimental activity.

In illustrative embodiments, the agonist of C3aR function comprises a peptide generally comprising an amino acid sequence having the formula:

X_{N}X₁X₂X₃X₄X₅X_{C}

wherein:

X_{N} is the N-terminus of the peptide and may comprise 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues, and suitably 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 amino acid residues, and more suitably 0, 1, 2, 3, 4, 5, 6, 7, 8, 9, 10, 11, or 12 amino acid residues. In exemplary embodiments, the sequence of X_{N} corresponds to residues 1 to 72 of the naturally-occurring human. C3a or corresponds to a fragment of residues 1 to 72 of the naturally-occurring human C3a, suitably residues 2 to 72, 3 to 72, 4 to 72, 5 to 72, 6 to 72, 7 to 72, 8 to 72, 9 to 72, 10 to 72, 11 to 72, 12 to 72, 13 to 72, 14 to 72, 15 to 72, 16 to 72, 17 to 72, 18 to 72, 19 to 72, 20 to 72, 21 to 72, 22 to 72, 23 to 72, 24 to 72, 25 to 72, 26 to 72, 27 to 72, 28 to 72, 29 to 72, 30 to 72, 31 to 72, 32 to 72, 33 to 72, 34 to 72, 35 to 72, 36 to 72, 37 to 72, 38 to 72, 39 to 72, 40 to 72, 41 to 72, 42 to 72, 43 to 72, 44 to 72, 45 to 72, 46 to 72, 47 to 72, 48 to 72, 49 to 72, 50 to 72, 51 to 72, 52 to 72, 53 to 72, 54 to 72, 55 to 72, 56 to 72, 57 to 72, 58 to 72, 59 to 72, 60 to 72, 61 to 72, 62 to 72, 63 to 72, 64 to 72, 65 to 72, 66 to 72, 67 to 72, 68 to 72, 69 to 72, 70 to 72, or 71 to 72 of the naturally-occurring human C3a;

X₁ is a hydrophobic amino acid residue or modified form thereof, and suitably a hydrophobic amino acid residue with an aliphatic side chain. In exemplary embodiments, X₁ is selected from tyrosine, valine, isoleucine, leucine, methionine, phenylalanine, tryptophan and modified forms thereof, suitably valine isoleucine, leucine, methionine, and modified forms thereof, more suitably leucine or methionine;

X₂ is a small amino acid residue or modified form thereof. In exemplary embodiments, X₂ is selected from glycine, serine, alanine, threonine, proline and modified forms thereof, suitably glycine, proline, or alanine;

X₃ is leucine or a modified form thereof (e.g., MeL or methyl leucine);

X₄ is a small amino acid residue or modified form thereof. In exemplary embodiments, X₄ is selected from glycine, serine, alanine, threonine, proline and modified forms thereof, suitably proline or alanine;

X₅ is arginine or a modified form thereof; and

X_{C} is the C-terminus of the peptide and may comprise 0, 1, 2, 3, 4, or 5 amino acid residues, suitably 0 (i.e., no) amino acid residues.

Peptides falling within the scope of the formula above include those described in Ember et al. 1991, including the peptide termed “EP141”, and those described in Takenaka et al. 2001, including LPLR [SEQ ID NO:4], PSYLPLPR [SEQ ID NO:5], and RPSYLPLPR [SEQ ID NO:6], and those described in Scully et al. 2010, including FLPLAR [SEQ ID NO:7], FIPLAR [SEQ ID NO:8], FWTLAR [SEQ ID NO:9], FLTLAR [SEQ ID NO:10], HLGLAR [SEQ ID NO:1 I], HLALAR [SEQ ID NO:12], YSFKDMP(MeL)aR [SEQ ID NO:13] (termed “EP67”), and YSFKPMPLaR [SEQ ID NO:14] (termed “EP54”), each of which are described further herein.

“Modified” amino acids that may be included in the peptides falling within the scope of the formula above can be gene-encoded amino acids which have been processed after translation of the gene, e.g., by the addition of methyl groups or derivatization through covalent linkages to other substituents or oxidation or reduction or other covalent modification. The classification into which the resulting modified amino acid falls will be determined by the characteristics of the modified form. For example, if lysine were modified by acylating the &amino group, the modified form would not be classed as basic but as polar/large. Certain commonly encountered modified amino acids, which are not encoded by the genetic code, include, for example, α-alanine (α-Ala), sarcosine (Sar), and α-aminoisobutyric acid (Aib), each of which is classified as a small amino acid, and t-butylalanine (t-BuA), t-butylglycine (tBu-G), N-methylisoleucine (N-MeIle), norleucine (Nle), cyclohexylalanine (Cha), phenylglycine (Phg), 2-napthylalanine (2-Nal), β-2-thienylalanine (Thi), and 1,2,3,4-tetrahydroisoquinoline-3-carboxlic acid (Tic), each of which is classified as a hydrophobic amino acid.

Contemplated modifications also include replacement of one or more amide linkages (—CO—NH) with another linkage which is an isostere such as —CH₂NH—, —CH₂S—, —CH₂CH₂—, —CH═CH— (cis and trans), —COCH₂—, —CH(OH)CH₂—, and —CH₂SO—. This replacement may be made by methods known in the art. The following references describe preparation of peptide analogues which include these alternative-linking moieties: Spatola A F, “Peptide Backbone Modifications” (general review) March 1983 Vega Data 1(3); Spatola A F, in “Chemistry and Biochemistry of Amino Acids Peptides and Proteins”, B Weinstein, eds., Marcel Dekker, New York, p. 267 (1983) (general review); Morley J S, 1980 Trends Pharm Sci (1980) pp. 463-468 (general review); Hudson D, et al., Int J Pept Prot Res (1979) 14:177-185 (—CH₂NH—, —CH₂CH₂—); Spatola A F, et al., Life Sci (1986) 38:1243-1249 (—CH₂—S); Hann M M, J Chem Soc Perkin Trans I (1982) 307-314 (—CH—CH—, cis and trans); Almiquist R G, et al., J Med Chem (1980) 23:1392-1398 (—COCH₂); Jennings-White C, et al., Tetrahedron Lett (1982) 23:2533 (—COCH₂—); Szelke M, et al., European Application EP 45665 (1982) CA:97:39405 (1982) (—CH(OH)CH₂—); Holladay M W, et al., Tetrahedron Lett (1983) 24:4401-4404 (—C(OH)CH₂—); and Hruby V J, Life Sci (1982) 31: 189-199 (—CH₂—S—).

Amino acid residues within the peptides, and particularly at the carboxy or amino-terminus, can also be modified by amidation, acetylation or substitution with other chemical groups which can, for example, change the solubility of the compounds without affecting their activity. Also, the protracted profile of the peptide compound can be improved by attaching a lipophilic substituent to any one or more amino acid residues, as for example, described in PCT international application WO 87-45329.

Examples of side chain modifications include modifications of amino groups such as acylation with acetic anhydride; acylation of amino groups with succinic anhydride and tetrahydrophthalic anhydride; amidination with methylacetimidate; carbamoylation of amino groups with cyanate; pyridoxylation of lysine with pyridoxal-5-phosphate followed by reduction with NaBH₄; reductive alkylation by reaction with an aldehyde followed by reduction with NaBH₄; and trinitrobenzylation of amino groups with 2,4,6-trinitrobenzene sulfonic acid (TNBS).

The carboxyl group may be modified by carbodiimide activation via O-acylisourea formation followed by subsequent derivatization, by way of example, to a corresponding amide.

The guanidine group of arginine residues may be modified by formation of heterocyclic condensation products with reagents such as 2,3-butanedione, phenylglyoxal and glyoxal.

Sulfhydryl groups may be modified by methods such as performic-acid oxidation to cysteic acid; formation of mercurial derivatives using 4-chloromercuriphenylsulphonic acid, 4-chloromercuribenzoate; 2-chloromercuri-4-nitrophenol, phenylmercury chloride, and other mercurials; formation of a mixed disulfides with other thiol compounds; reaction with maleimide, maleic anhydride or other substituted maleimide; carboxymethylation with iodoacetic acid or iodoacetamide; and carbamoylation with cyanate at alkaline pH.

Tryptophan residues may be modified, for example, by alkylation of the indole ring with 2-hydroxy-5-nitrobenzyl bromide or sulfonyl halides or by oxidation with N-bromosuccinimide.

Tyrosine residues may be modified by nitration with tetranitromethane to form a 3-nitrotyrosine derivative.

The imidazole ring of a histidine residue may be modified by N-carbethoxylation with diethylpyrocarbonate or by alkylation with iodoacetic acid derivatives.

Examples of incorporating unnatural amino acids and derivatives during peptide synthesis include but are not limited to, use of 4-amino butyric acid, 6-aminohexanoic acid, 4-amino-3-hydroxy-5-phenylpentanoic acid, 4-amino-3-hydroxy-6-methylheptanoic acid, t-butylglycine, norleucine, norvaline, phenylglycine, omithine, sarcosine, 2-thienyl alanine and/or D-isomers of amino acids. A list of unnatural (or modified) amino acids contemplated by the present invention is shown in Table 2 below.

TABLE 2
Non-Conventional (or Modified) Amino Acids
NON-CONVENTIONAL AMINO ACIDS

| | |
|---|---|
| α-aminobutyric acid | L-N-methylalanine |
| α-amino-α-methylbutyrate | L-N-methylarginine |
| aminocyclopropane-carboxylate | L-N-methylasparagine |
| aminoisobutyric acid | L-N-methylaspartic acid |
| aminonorbornyl-carboxylate | L-N-methylcysteine |
| cyclohexylalanine | L-N-methylglutamine |
| cyclopentylalanine | L-N-methylglutamic acid |
| L-N-methylisoleucine | L-N-methylhistidine |
| D-alanine | L-N-methylleucine |
| D-arginine | L-N-methyllysine |
| D-aspartic acid | L-N-methylmethionine |
| D-cysteine | L-N-methylnorleucine |
| D-glutamate | L-N-methylnorvaline |
| D-glutamic acid | L-N-methylornithine |
| D-histidine | L-N-methylphenylalanine |
| D-isoleucine | L-N-methylproline |
| D-leucine | L-N-medlylserine |
| D-lysine | L-N-methylthreonine |
| D-methionine | L-N-methyltryptophan |
| D-ornithine | L-N-methyltyrosine |
| D-phenylalanine | L-N-methylvaline |
| D-proline | L-N-methylethylglycine |
| D-serine | L-N-methyl-t-butylglycine |
| D-threonine | L-norleucine |
| D-tryptophan | L-norvaline |
| D-tyrosine | α-methyl-aminoisobutyrate |
| D-valine | α-methyl-γ-aminobutyrate |
| D-α-methylalanine | α-methylcyclohexylalanine |
| D-α-methylarginine | α-methylcylcopentylalanine |
| D-α-methylasparagine | α-methyl-α-napthylalanine |
| D-α-methylaspartate | α-methylpenicillamine |
| D-α-methylcysteine | N-(4-aminobutyl)glycine |
| D-α-methylglutamine | N-(2-aminoethyl)glycine |
| D-α-methylhistidine | N-(3-aminopropyl)glycine |
| D-α-methylisoleucine | N-amino-α-methylbutyrate |
| D-α-methylleucine | α-napthylalanine |
| D-α-methyllysine | N-benzylglycine |
| D-α-methylmethionine | N-(2-carbamylediyl)glycine |
| D-α-methylornithiine | N-(carbamylmethyl)glycine |
| D-α-methylphenylalanine | N-(2-carboxyethyl)glycine |
| D-α-methylproline | N-(carboxymethyl)glycine |
| D-α-methylserine | N-cyclobutylglycine |
| D-α-methylthreonine | N-cycloheptylglycine |
| D-α-methyltryptophan | N-cyclohexylglycine |
| D-α-methyltyrosine | N-cyclodecylglycine |
| L-α-methylleucine | L-α-methyllysine |
| L-α-methylmethionine | L-α-methylnorleucine |
| L-α-methylnorvatine | L-α-methylornithine |
| L-α-methylphenylalanine | L-α-methylproline |
| L-α-methylserine | L-α-methylthreonine |
| L-α-methyltryptophan | L-α-methyltyrosine |
| L-α-methylvaline | L-N-methylhomophenylalanine |
| N-(N-(2,2-diphenylethyl | N-(N-(3,3-diphenylpropyl |
| carbamylmethyl)glycine | carbamylmethyl)glycine |
| 1-carboxy-1-(2,2-diphenyl-ethyl | |
| amino)cyclopropane | |

Peptides suitable for use in the present invention may be prepared in recombinant form using standard protocols as, for example, described in Sambrook et al. MOLECULAR CLONING. A LABORATORY MANUAL (Cold Spring Harbor Press, 1989), in particular Sections 16 and 17; Ausubel et al. CURRENT PROTOCOLS IN MOLECULAR BIOLOGY (John Wiley & Sons, Inc. 1994-1998), in particular Chapters 10 and 16; and Coligan et al. CURRENT PROTOCOLS IN PROTEIN SCIENCE (Jon Wiley & Sons, Inc. 1995-1997), in particular Chapters 1, 5, and 6. Typically, the peptide may be prepared by a procedure including the steps of (a) providing an expression vector from which the peptide is expressible; (b) introducing the vector into a suitable host cell; (c) culturing the host cell to express recombinant peptide from the vector; and (d) isolating the recombinant peptide. Alternatively, the peptide can be synthesized using solution synthesis or solid phase synthesis as described, for example, by Atherton and Sheppard in SOLID PHASE PEPTIDE SYNTHESIS: A PRACTICAL APPROACH (IRL Press at Oxford University, Oxford, England, 1998) or by Roberge et al. (1995 Science 269: 202). Syntheses may employ, for example, either t-butyloxycarbonyl (t-Boc) or 9-fluorenylmethyloxycarbonyl (Fmoc) chemistries (see Chapter 9.1 of Coligan et al. supra; Stewart and Young, 1984, SOLID PHASE PEPTIDE SYNTHESIS, 2″ ed. Pierce Chemical Co., Rockford, Ill., 1994; and Atherton and Shephard, supra).

Suitably, C3 or C3 convertase is the subject of the targeting. For example, the C3aR agonist may agonize or otherwise activate C3 and/or cleavage of C3 into C3a and C3b.

In still other embodiments, the subject of the targeting is a component of post-receptor signal transduction.

The present invention also contemplates the use in the above method of gene or expression product agonists identified according to methods described herein.

### 3.1 Illustrative Small Organic Molecules Include

(A) The arginine-containing compounds as described in Denonne et al. 2007 (Part 1), demonstrated to have affinity for C3aR and to have C3aR agonist activity, including the biaryl-substituted arginine compounds and the triarylmethine-substituted arginine compounds, including the following:

(i) “Compound 12” having the structure:

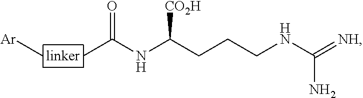

wherein the aryl group comprises 3-chlorophenyl, and

the linker comprises 2,5-furyl;

(ii) “Compound 23” having the structure:

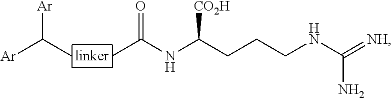

wherein the diarylmethine group comprises 3-fluorophenyl, and

the linker comprises 2,5-furyl;

(iii) “Compound 24” having the structure:

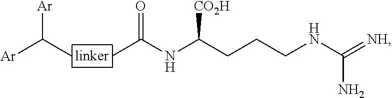

wherein the diarylmethine group comprises 3-chlorophenyl, and

the linker comprises 2,5-furyl; and

(iv) “Compound 25” having the structure:

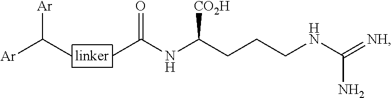

wherein the diarylmethine group comprises 4-chlorophenyl, and

the linker comprises 2,5-furyl.

Methods of producing the arginine-containing compounds, and testing the compounds for C3aR affinity and agonist activity using the functional assay are described in Denonne et al. 2007 (Part 1).

(B) The amino-piperidine derivatives as described in Denonne et al. 2007 (Part 2), having the structure:

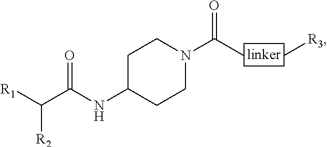

and demonstrated to have affinity for C3aR and to have C3aR agonist activity, including the following:

(i) “Compound 7” wherein R1 comprises phenyl, R2 comprises cyclohexyl, the linker comprises C₂H₄, and R3 comprises 3-pyridyl. Compound 7 comprises two separate enantiomers termed “Compound 7A” and “Compound 7B” where the R2 group in each compound comprises a different epimer of cyclohexyl;

(ii) “Compound 22” wherein R1 comprises 3-chlorophenyl, R2 comprises cyclohexyl, the linker comprises C₂H₄, and R3 comprises 3-pyridyl;

(iii) “Compound 25” wherein R1 comprises 3,4,5-trifluorophenyl, R2 comprises cyclohexyl, the linker comprises C₂H₄, and R3 comprises 3-pyridyl;

(iv) “Compound 27” wherein R1 comprises 2-naphthyl, R2 comprises cyclohexyl, the linker comprises C₂H₄, and R3 comprises 3-pyridyl;

(v) “Compound 30” wherein R1 comprises phenyl, R2 comprises cyclohexyl, the linker comprises C₂H₄, and R3 comprises 2-fluoro-3-pyridyl.

These compounds, methods for their preparation and their biological activity are disclosed in Denonne et al. 2007 (Part 2).

(C) The compound benzeneacetamide,α-cyclohexyl-N-[1-[1-oxo-3-(3-pyridinyl)propyl]-4-piperidinyl]-, having the structure:

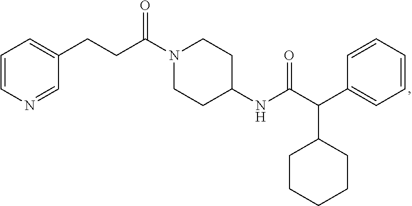 [SMILES: O=C(NC1CCN(C(=O)CCc2cccnc2)CC1)C(c1ccccc1)C1CCCCC1]

which is α-cyclohexyl-N-[1-[1-oxo-3-(3-pyridinyl)propyl]-4-piperidinyl]-benzeneacetamide sold by Sigma-Aldrich under the name “C4494”. Derivatives and stereoisomers of this compound are also contemplated.

(D) The compound 5-(diaminomethylideneamino)-2-[[2-(2,2-diphenylethoxy)acetyl]amino]pentanoic acid having the structure:

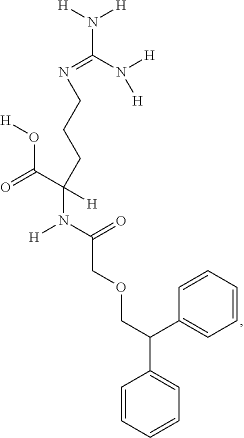 [SMILES: NC(N)=NCCCC(NC(=O)COCC(c1ccccc1)c1ccccc1)C(=O)O]

which is also termed “SB290157”, “AC1L1JR3” and “CID5167”. This compound, methods for its preparation and its biological activity are disclosed in Mathieu et al. 2005. Derivatives and stereoisomers of this compound are also contemplated.

### 3.2 Illustrative Aptamers, Peptides, Peptidomimetics, Polypeptides, and Proteins Include

(A) The peptide termed C3a that is produced in the body upon cleavage of C3 by C3 convertase (also known as C4b2a or C3bBb) into C3a and C3b, and that activates or agonizes the C3a receptor (C3aR). In some embodiments, this peptide may be isolated from a naturally occurring source, such as a biological sample from an individual, including human (sequence: SVQLTEKRMDKVGKYPICELRICCCEDGMRENPMRFSCQRRTRFISLGEACKKVEL DCCNYITELRRQHARASHLGLAR [SEQ ID NO:1]) or other animal origin, including but not limited to C3a from companion animals, livestock, Sus scrofa (e.g., GenBank accession no. P01025), and Rattus norvegicus (e.g., GenBank accession no. AAG00532). In some other embodiments, the peptide may be synthesized using art-known methods, for example a recombinant form of human C3a comprising 78 amino acid residues is described in Fukuoka et al. 1991 and in GenBank accession no. AAA73037.

(B) Aptamers, peptide analogues, peptide variants, or peptidomimetics of C3a which are C3a receptor agonists, including C3a analogues where the analogues comprise modifications to the N-terminal and/or C-terminal regions of C3a, derivatives of the analogues, and dimeric forms of the analogues and derivatives, including those C3a or C3a analogues with amino-terminal hydrophobic groups such as Fmoc attached, as originally introduced by Gerardy-Schahn et al. 1988.

Such aptamers, peptide analogues, peptide variants or peptidomimetics include those having a sequence and/or structure based on the finding that a region within the C-terminal of C3a is essential for binding to C3aR and agonizing C3aR.

In Ember et al. 1991, the authors state that residues 63-77 in human C3a contains the essential functional site of the molecule, although variation within the natural sequence was permitted. Ember et al. 1991 specifically describe the “superagonist” termed “EP141”, an agonist of C3aR function which is the peptide having the sequence WWGKKYRASKLGLAR [SEQ ID NO:3]. This peptide was designed by incorporating two tryptophanyl residues at the N-terminal end of an analogue of the 15-mer ‘essential functional site’ region of C3a (i.e., an analogue of residues 63-77 of human C3a with a number of naturally-occurring residues replaced with other residues). This peptide was shown to be a highly potent and selective C3aR agonist. The design, synthesis and testing of this peptide and a number of other suitable peptides comprising agonists of C3aR function is described in Ember et al. 1991, each of which is suitable for use in the present invention.

In Takenaka et al. 2001, the authors state that the C-terminus LPLPR sequence of C3a is essential for C3aR activation and thus contemplate peptides having a sequence and/or structure with C3aR agonist activity based on this finding, including the following exemplary peptides:

(i) a peptide comprising the amino acid sequence LPLPR [SEQ ID NO:4],

(ii) a peptide comprising the amino acid sequence PSYLPLPR [SEQ ID NO:5],

(iii) a peptide comprising the amino acid sequence RPSYLPLPR [SEQ ID NO:6], and

modified forms thereof, including chemical modification by acetylation, or single or multiple amino acid replacement, insertion, or deletion.

These C3aR agonists, methods of their preparation, and methods of testing their activity are described in Takenaka et al. 2001, and each is suitable for use in the present invention.

Scully et al. 2010 also discuss the C-terminal region of C3a noting that the C-terminal octapeptide region of C3a is implicated as the effector domain required for triggering receptor activation. Scully et al. 2010 describe their research into agonists of C3aR function, including peptide agonists they derived based on structure-activity relationships of peptides based on the C-terminus of C3a. The authors identify four specific hexapeptides with potent and selective C3aR agonist activity, being:

(i) a hexapeptide comprising the amino acid sequence FLPLAR [SEQ ID NO:7];

(ii) a hexapeptide comprising the amino acid sequence FIPLAR [SEQ ID NO:8];

(iii) a hexapeptide comprising the amino acid sequence FWTLAR [SEQ ID NO:9]; and

(iv) a hexapeptide comprising the amino acid sequence FLTLAR [SEQ ID NO:10].

These four hexapeptide C3aR agonists, methods of their preparation, and methods of testing their activity are described in Scully et al. 2001, and each is suitable for use in the present invention.

The authors of Scully et al. 2010 also draw some conclusions about the requirements of human C3aR for potent and selective hexapeptide ligands including the following: (i) the C-terminal Leu3-Ala-2-Arg1 motif is important for agonist activity, with changes to this region leading to significant reductions in agonist potency; (ii) threonine at the fourth position gave the most potent compounds, although other amino acids with small side chains (serine, alanine, proline, glycine) were not that much less potent; (iii) the most potent agonists had tryptophan, norleucine, or leucine at the fifth position; (iv) only ligands with phenylalanine at the sixth or N-terminal position were potent agonists; (v) replacing leucine at the third position of short peptide agonist FLTLAR [SEQ ID NO:10] with a bulkier group leads to compound FLTChaAR [SEQ ID NO:15], which is a functional antagonist of both C3a and the short peptide agonist FWTLAR [SEQ ID NO:9]. It is contemplated that compounds adhering to these conclusions may be made and used in accordance with the present invention.

Other suitable compounds having a sequence and/or structure based on the finding that the C-terminus of C3a is essential for C3aR activation are also contemplated.

(C) Peptide compounds comprising the amino acid sequence HLGLAR, modified forms thereof, including chemical modification by acetylation, or single or multiple amino acid replacement, insertion, or deletion.

(D) Peptide compounds comprising the amino acid sequence HLALAR, modified forms thereof, including chemical modification by acetylation, or single or multiple amino acid replacement, insertion, or deletion.

(E) The conformationally constrained agonist of C3aR function termed “EP67” having the amino acid sequence: YSFKDMP(MeL)aR [SEQ ID NO:13]. The design, synthesis and testing of this peptide is described in Vogen et al. 2001.

(F) The peptide compound termed “EP54” having the amino acid sequence: YSFKPMPLaR [SEQ ID NO:14].

(G) Antibodies that bind to C3aR and agonize C3aR function. Suitably the antibodies are monoclonal or polyclonal antibodies. Suitable antibodies can be produced and screened by methods well known in the art.

### 3.3 Illustrative Other Agonists of C3aR Function

Many other suitable agonists of C3aR function are known in the art in addition to those specifically described above.

These include an agonist of Farnesoid X receptor (FXR) function which was shown to increase plasma C3 protein concentration as described in Li et al. 2005.

### 4. Identification of Agents that Agonize C3aR Function

The present invention also provides methods for identifying agents that agonize C3aR function for preventing or treating an acute inflammatory condition.

The methods may comprise screening for an agent that modulates the expression of a gene or the level and/or functional activity of an expression product of that gene, wherein the gene is selected from a gene encoding a component of the complement system associated with C3aR function, a gene whose expression product modulates directly or indirectly the expression of a gene encoding a component of the complement system associated with C3aR function, and a gene whose expression product modulates directly or indirectly the expression product of a gene encoding a component of the complement system associated with C3aR function.

Illustrative methods may comprise contacting a sample of cells expressing C3aR with a test agent. Optionally, the level of binding between the C3aR and the test agent is first measured to determine whether the test agent binds (i.e., interacts directly) with the C3aR. A detected increase in level of activity of C3aR relative to a reference or control level in the absence of the test agent, indicates that the agent agonizes C3aR function and that it is useful for preventing or treating an acute inflammatory condition.

In some embodiments, the methods of screening for a C3aR agonist comprise: (a) contacting a composition comprising a C3aR polypeptide in the presence and absence of a test agent; (b) measuring C3aR activation in the presence and absence of the test agent; and (c) selecting as a C3aR agonist a test agent that induces C3aR activation. In one variation, the measuring step comprises: (i) measuring binding between the test agent and the C3aR polypeptide; and (ii) measuring C3aR internalization, wherein a test agent that binds C3aR and induces C3aR internalization is identified as a C3aR agonist. In another variation, the measuring step comprises: (i) measuring binding between the test agent and the C3aR polypeptide; and (ii) measuring C3aR signaling, wherein a test agent that binds C3aR and induces C3aR intracellular signaling is identified as a C3aR agonist. A variety of techniques for measuring receptor signaling are known in the art.

Any suitable method to detect an increase in level of activity of C3aR relative to a reference or control level may be used, including those assays which measure an output from the cells expressing C3aR, non-limiting examples of which include neutrophils, mast cells, astrocytes, adipocytes, macrophages and C3aR-expressing cell lines. The output may be intracellular calcium mobilization (e.g., a hallmark of G-protein coupled receptor activation) or granular enzyme release (e.g., (3-glucuronidase). Suitable output assays are described in Scully et al. 2010. In illustrative embodiments, the method may comprise the use of the screening method reviewed in Motthaekis and Ohler, 2000. In this screening method, a cell line expressing a G-protein coupled receptor (C3aR for the purposes of the present invention) and aequorin is employed. Such cell lines are known in the art, for example, in Sheu et al. 1993 and Button and Brownstein, 1993. The cells are incubated with coelenterazine, which is a co-factor of aequorin. During the incubation, coelenterazine enters the cell (it is lipophilic and readily crosses the cell membrane) and conjugated with apoaequorin to form aequorin, which is the active form of the enzyme. When the cells are then exposed to a test agent, if the test agent is an agonist of C3aR function then it will activate the C3a receptor leading to an increase in intracellular calcium levels. This increase leads to the activation of the catalytic activity of aequorin which oxidizes coelenterazine and yields apoaequorin, coelenteramide, CO₂ and light. The intensity of light emission is proportional to the increase in intracellular calcium (Rizutto et al. 1995). Accordingly, measurement of light emission in this system following addition of a test agent can be used to determine its ability to agonize C3aR function. Methods in accordance with this screening method were employed to identify the arginine-containing compounds that are agonists of C3aR function as described in Denonne et al. 2007 (Part 1) and the amino-piperidine derivatives that are agonists of C3aR function as described in Denonne et al. 2007 (Part 2), and these compounds are discussed in the previous section.

Another suitable method comprises contacting a sample of cells expressing C3aR (e.g., neutrophils, mast cells, astrocytes, adipocytes, macrophages and C3aR-expressing cell lines) with C3a and a test agent. A detected increase in level of binding between the C3aR and the C3a relative to a reference or control level in the absence of the test agent, indicates that the agent agonizes C3aR function and that it is useful for preventing or treating an acute inflammatory condition.

Yet another suitable method comprises contacting a preparation with a test agent, wherein the preparation comprises (i) a polypeptide comprising an amino acid sequence corresponding to at least a biologically active fragment of a polypeptide component of the complement system associated with C3aR function, or to a variant or derivative thereof; or (ii) a polynucleotide comprising at least a portion of a genetic sequence (e.g. a transcriptional control element such as a promoter or a cis-acting sequence) that regulates a nucleotide sequence that encodes at least a biologically active fragment of a polypeptide component of the complement system associated with C3aR function, or a variant or derivative thereof, which is operably linked to a reporter gene; or (iii) a polynucleotide comprising a nucleotide sequence that encodes a polypeptide according to (i). A detected change in the level and/or functional activity of the polypeptide component, or an expression product of the reporter gene, relative to a reference and/or control level or functional activity in the absence of the test agent, indicates that the agent modulates C3aR function and that it is useful for preventing or treating an acute inflammatory condition. Suitably the method comprises identifying agents that agonize C3aR function, wherein an increase in the level and/or functional activity of the polypeptide component (e.g., C3, C3aR), or an expression product of the reporter gene, relative to a reference and/or control level or functional activity in the absence of the test agent, indicates that the agent agonizes C3aR function and that it is useful for preventing or treating an acute inflammatory condition.

Yet another suitable method comprises a first sample comprising cells (e.g., neutrophils, mast cells, astrocytes, adipocytes, macrophages and C3aR-expressing cell lines) expressing C3aR that are contacted with a test agent, and a second sample comprising either cells that do not express C3aR (e.g., C3aR^{-/-}) that are contacted with a test agent, or comprising cells expressing C3aR that are contacted with a vehicle or control. The samples of cells may comprise in vitro samples (e.g., cell lines) or an in vivo model (e.g., animal model including mouse model). A detected difference in cell output (e.g., intracellular calcium mobilization or granular enzyme release) between the first sample and the second sample indicates that the test agent modulates C3aR function. Suitably the method comprises identifying agents that agonize C3aR function, wherein an increase in the measured cell output in the first sample relative to the second sample, indicates that the agent agonizes C3aR function and that it is useful for preventing or treating an acute inflammatory condition. Alternatively, a detected difference in another measured input (e.g., response to induced acute inflammatory condition, level or activity level of neutrophils measured in any suitable way including neutrophil mobilization and neutrophil infiltration of tissue) between the first sample and the second sample indicates that the test agent modulates C3aR function. Suitably the method comprises identifying agents that agonize C3aR function, wherein the measured output indicates a response to the acute inflammatory condition (e.g., reduced inflammation, reduced level or activity of neutrophils, reduced neutrophil mobilization, reduced neutrophil infiltration of tissue etc.), and thus indicates that the agent agonizes C3aR function and that it is useful for preventing or treating an acute inflammatory condition.

Candidate test agents encompass numerous chemical classes. Candidate organic molecules comprise functional groups necessary for structural interaction with components of the complement system associated with C3aR function, including C3aR, including proteins, particularly by way of hydrogen bonding, and typically include at least an amine, carbonyl, hydroxyl or carboxyl group, suitably at least two of the functional chemical groups. The candidate test agent often comprises cyclical carbon or heterocyclic structures or aromatic or polyaromatic structures substituted with one or more of the above functional groups. Candidate test agents are also found among biomolecules including, but not limited to: peptides, saccharides, fatty acids, steroids, purines, pyrimidines, derivatives, structural analogues or combinations thereof.

In some embodiments, small molecules are used as candidate test agents because such molecules are more readily absorbed after oral administration, have fewer potential antigenic determinants, or are more likely to cross the cell membrane than larger, protein-based pharmaceuticals. Small organic molecules may also have the ability to gain entry into an appropriate cell and affect the expression of a gene (e.g., by interacting with the regulatory region or transcription factors involved in gene expression); or affect the activity of a gene by inhibiting or enhancing the binding of accessory molecules.

Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogues.

Screening may also be directed to known pharmacologically active compounds and chemical analogues thereof.

Screening candidate test agents according to the invention can be achieved by any suitable method. For example, the method may include contacting a cell expressing a polynucleotide corresponding to gene encoding a component of the complement system associated with C3aR function, with an agent suspected of having the modulatory activity and screening for the modulation of the level or functional activity of a protein encoded by the polynucleotide, or the modulation of the level of a transcript encoded by the polynucleotide, or the modulation of the activity or expression of a downstream cellular target of the protein or of the transcript (hereafter referred to as target molecules). Detecting such modulation can be achieved utilizing techniques including, but not restricted to, ELISA, cell-based ELISA, inhibition ELISA, Western blots, immunoprecipitation, slot or dot blot assays, immunostaining, RIA, scintillation proximity assays, fluorescent immunoassays using antigen-binding molecule conjugates or antigen conjugates of fluorescent substances such as fluorescein or rhodamine, Ouchterlony double diffusion analysis, immunoassays employing an avidin-biotin or a streptavidin-biotin detection system, and nucleic acid detection assays including reverse transcriptase polymerase chain reaction (RT-PCR).

It will be understood that a polynucleotide from which a target molecule of interest is regulated or expressed may be naturally occurring in the cell which is the subject of testing or it may have been introduced into the host cell for the purpose of testing. Further, the naturally-occurring or introduced polynucleotide may be constitutively expressed—thereby providing a model useful in screening for agents which down-regulate expression of an encoded product of the sequence wherein the down regulation can be at the nucleic acid or expression product level—or may require activation—thereby providing a model useful in screening for agents that up-regulate expression of an encoded product of the sequence. Further, to the extent that a polynucleotide is introduced into a cell, that polynucleotide may comprise the entire coding sequence which codes for a target protein or it may comprise a portion of that coding sequence (e.g., a binding domain) or a portion that regulates expression of a product encoded by the polynucleotide (e.g., a promoter). For example, the promoter that is naturally associated with the polynucleotide may be introduced into the cell that is the subject of testing. In this regard, where only the promoter is utilized, detecting modulation of the promoter activity can be achieved, for example, by operably linking the promoter to a suitable reporter polynucleotide including, but not restricted to, green fluorescent protein (GFP), luciferase, β-galactosidase and catecholamine acetyl transferase (CAT). Modulation of expression may be determined by measuring the activity associated with the reporter polynucleotide.

In another example, the subject of detection could be a downstream regulatory target of the target molecule, rather than the target molecule itself or the reporter molecule operably linked to a promoter of a gene encoding a product the expression of which is regulated by the target protein.

These methods provide a mechanism for performing high throughput screening of putative modulatory agents such as proteinaceous or non-proteinaceous agents comprising synthetic, combinatorial, chemical and natural libraries. These methods will also facilitate the detection of agents which bind either the polynucleotide encoding the target molecule or which modulate the expression of an upstream molecule, which subsequently modulates the expression of the polynucleotide encoding the target molecule. Accordingly, these methods provide a mechanism of detecting agents that either directly or indirectly modulate the expression or activity of a target molecule according to the invention.

In a series of embodiments, the present invention provides assays for identifying small molecules or other compounds (i.e., modulatory agents) which are capable of inducing or inhibiting the level and/or functional activity of target molecules according to the invention. The assays may be performed in vitro using non-transformed cells, immortalized cell lines, or recombinant cell lines. In addition, the assays may detect the presence of increased or decreased expression of genes or production of proteins on the basis of increased or decreased mRNA expression (using, for example, the nucleic acid probes disclosed herein), increased or decreased levels of protein products (using, for example, the antigen binding molecules disclosed herein), or increased or decreased levels of expression of a reporter gene (e.g., GFP, β-galactosidase or luciferase) operably linked to a target molecule-related gene regulatory region in a recombinant construct.

Thus, for example, one may culture cells which produce a particular target molecule and add to the culture medium one or more test compound's. After allowing a sufficient period of time (e.g., 6-72 hours) for the compound to induce or inhibit the level or functional activity of the target molecule, any change in the level from an established baseline may be detected using any of the techniques described above and well known in the art. Using suitable nucleic acid probes or antigen-binding molecules, detection of changes in the level and or functional activity of a target molecule, and thus identification of the compound as agonist or antagonist of the target molecule, requires only routine experimentation.

In some embodiments, recombinant assays are employed in which a reporter gene encoding, for example, GFP, β-galactosidase or luciferase is operably linked to the 5′ regulatory regions of a target molecule related gene. Such regulatory regions may be easily isolated and cloned by one of ordinary skill in the art. The reporter gene and regulatory regions are joined in-frame (or in each of the three possible reading frames) so that transcription and translation of the reporter gene may proceed under the control of the regulatory elements of the target molecule related gene. The recombinant construct may then be introduced into any appropriate cell type, suitably mammalian cells, most suitably human cells. The transformed cells may be grown in culture and, after establishing the baseline level of expression of the reporter gene, test compounds may be added to the medium. The ease of detection of the expression of the reporter gene provides for a rapid, high throughput assay for the identification of agonists or antagonists of the target molecules of the invention.

Compounds identified by this method will have potential utility in modifying the expression of target molecule related genes in vivo. These compounds may be further tested in the animal models to identify those compounds having the most potent in vivo effects. In addition, as described above with respect to small molecules having target polypeptide binding activity, these molecules may serve as “lead compounds” for the further development of pharmaceuticals by, for example, subjecting the compounds to sequential modifications, molecular modeling, and other routine procedures employed in rational drug design.

In other embodiments, methods of identifying agents that agonize the complement system associated with C3aR function are provided in which a purified preparation of a component of the complement system associated with C3aR function is incubated in the presence and absence of a candidate agent under conditions in which the component is active, and the level of activity is measured by a suitable assay. For example, an agonist of C3aR function can be identified by measuring the ability of a candidate agent to increase C3a-C3aR binding. An agent tests positive if it agonizes or increases this activity.

In still other embodiments, random peptide libraries consisting of all possible combinations of amino acids attached to a solid phase support may be used to identify peptides that are able to bind to a target molecule or to a functional domain thereof. Identification of molecules that are able to bind to a target molecule may be accomplished by screening a peptide library with a recombinant soluble target molecule. The target molecule may be purified, recombinantly expressed or synthesized by any suitable technique. Such molecules may be conveniently prepared by a person skilled in the art using standard protocols as for example described in Sambrook, et al., (1989, supra) in particular Sections 16 and 17; Ausubel et al., (“Current Protocols in Molecular Biology”, John Wiley & Sons Inc, 1994-1998), in particular Chapters 10 and 16; and Coligan et al., (“Current Protocols in Immunology”, (John Wiley & Sons, Inc, 1995-1997), in particular Chapters 1, 5 and 6. Alternatively, a target polypeptide according to the invention may be synthesized using solution synthesis or solid phase synthesis as described, for example, in Chapter 9 of Atherton and Shephard (supra) and in Roberge et al. (1995).

To identify and isolate the peptide/solid phase support that interacts and forms a complex with a target molecule, suitably a target polypeptide, it may be necessary to label or “tag” the target polypeptide. The target polypeptide may be conjugated to any suitable reporter molecule, including enzymes such as alkaline phosphatase and horseradish peroxidase and fluorescent reporter molecules such as fluorescein isothiocynate (FITC), phycoerythrin (PE) and rhodamine. Conjugation of any given reporter molecule, with target polypeptide, may be performed using techniques that are routine in the art. Alternatively, target polypeptide expression vectors may be engineered to express a chimeric target polypeptide containing an epitope for which a commercially available antigen-binding molecule exists. The epitope specific antigen-binding molecule may be tagged using methods well known in the art including labeling with enzymes, fluorescent dyes or colored or magnetic beads.

For example, the “tagged” target polypeptide conjugate is incubated with the random peptide library for 30 minutes to one hour at 22° C. to allow complex formation between target polypeptide and peptide species within the library. The library is then washed to remove any unbound target polypeptide. If the target polypeptide has been conjugated to alkaline phosphatase or horseradish peroxidase the whole library is poured into a petri dish containing a substrate for either alkaline phosphatase or peroxidase, for example, 5-bromo-4-chloro-3-indoyl phosphate (BCIP) or 3,3′,4,4″-diarrmobenzidine (DAB), respectively. After incubating for several minutes, the peptide/solid phase-target polypeptide complex changes color, and can be easily identified and isolated physically under a dissecting microscope with a micromanipulator. If a fluorescently tagged target polypeptide has been used, complexes may be isolated by fluorescent activated sorting. If a chimeric target polypeptide having a heterologous epitope has been used, detection of the peptide/target polypeptide complex may be accomplished by using a labeled epitope specific antigen-binding molecule. Once isolated, the identity of the peptide attached to the solid phase support may be determined by peptide sequencing.

Illustrative methods and assays of screening for C3aR agonists are described for example in U.S. Patent Application Publication No. 20070054325.

In accordance with the present invention, the agents identified using the methods broadly described above are useful for preventing or treating an acute inflammatory condition in an individual.

### 5. Methods of Producing Agents Comprising an Agonist of C3aR Function

The present invention also provides methods of producing an agent comprising an agonist of C3aR function for preventing or treating an acute inflammatory condition in an individual. These methods generally comprise: testing a test agent as broadly described above; and synthesizing the agent on the basis that it tests positive for agonizing C3aR function.

Suitably, the method further comprises derivatizing the agent, and optionally formulating the derivatized agent with a pharmaceutically acceptable carrier or diluent, to improve the efficacy of the agent for treating or preventing an acute inflammatory condition. Suitable methods are known in the art.

### 6. Formulations and Administration of Agonists of C3aR Function

In accordance with the present invention, it is proposed that agents that comprise agonists of C3aR function are useful for the prevention or treatment of acute inflammatory conditions in an individual. In specific embodiments, the agents are administered to the individual after identifying that the individual has or is at risk of developing an acute inflammatory condition.

The agonist of C3aR function may comprise any suitable agonist of C3aR function including those described herein, and those agents comprising an agonist of C3aR function identified using the methods broadly described above.

Such agents can be administered to an individual either by themselves, or in pharmaceutical compositions where they are mixed with a suitable pharmaceutically acceptable carrier or diluent.

The agents of the present invention may be conjugated with biological targeting agents which enable their activity to be restricted to particular cell types. Such biological-targeting agents include substances which are immuno-interactive with cell-specific surface antigens.

The agents may be formulated and administered systemically or locally. Techniques for formulation and administration may be found in “Remington's Pharmaceutical Sciences,” Mack Publishing Co., Easton, Pa., latest edition. Suitable routes may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For injection, the agents may be formulated in aqueous solutions, suitably in physiologically compatible buffers such as Hanks' solution, Ringer's solution, or physiological saline buffer. For transmucosal administration, penetrants appropriate to the barrier to be permeated are used in the formulation. Such penetrants are generally known in the art. Intra-muscular and subcutaneous injection is also contemplated.

The agents can be formulated readily using pharmaceutically acceptable carriers or diluents well known in the art into dosages suitable for oral administration. Such carriers or diluents enable the compounds of the invention to be formulated in dosage forms such as tablets, pills, capsules, liquids, gels, syrups, slurries, suspensions and the like, for oral ingestion by a patient to be treated. These carriers or diluents may be selected from sugars, starches, cellulose and its derivatives, malt, gelatin, talc, calcium sulfate, vegetable oils, synthetic oils, polyols, alginic acid, phosphate buffered solutions, emulsifiers, isotonic saline, and pyrogen-free water.

Pharmaceutical compositions suitable for use in the present invention include compositions wherein the active ingredients are contained in an effective amount to achieve its intended purpose. The dose of agent administered to an individual should be sufficient to effect a beneficial response in the individual over time such as reducing or otherwise ameliorating in an individual the symptoms of an acute inflammatory condition or the symptoms of a disease or condition that causes or is otherwise associated with an acute inflammatory condition. The quantity of the agent(s) to be administered may depend on the subject to be treated inclusive of the age, sex, weight and general health condition thereof. In this regard, precise amounts of the agent(s) for administration will depend on the judgment of the practitioner. In determining the effective amount of the drug to be administered, the physician may evaluate the characteristics of the patient, their response to the drug and the safety profile of the drug. In any event, those of skill in the art may readily determine suitable dosages of the agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or other components which increase the solubility of the compounds to allow for the preparation of highly concentrated solutions.

Pharmaceutical preparations for oral use can be obtained by combining the active compounds with solid carriers, optionally grinding a resulting mixture, and processing the mixture of granules, after adding suitable auxiliaries, if desired, to obtain tablets or dragee cores. Suitable carriers are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, or polyvinylpyrrolidone (PVP). If desired, disintegrating agents may be added, such as the cross-linked polyvinyl pyrrolidone, agar, or alginic acid or a salt thereof such as sodium alginate. Such compositions may be prepared by any of the methods of pharmacy but all methods include the step of bringing into association one or more drugs as described above with the carrier or diluent which constitutes one or more necessary ingredients. In general, the pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Dragee cores are provided with suitable coatings. For this purpose, concentrated sugar solutions may be used, which may optionally contain gum arabic, talc, polyvinyl pyrrolidone, carbopol gel, polyethylene glycol, or titanium dioxide, lacquer solutions, and suitable organic solvents or solvent mixtures. Dyestuffs or pigments may be added to the tablets or dragee coatings for identification or to characterize different combinations of active compound doses.

Pharmaceuticals which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

Dosage forms of the agents may also include injecting or implanting controlled releasing devices designed specifically for this purpose or other forms of implants modified to act additionally in this fashion. Controlled release of an agent of the invention may be effected by coating the same, for example, with hydrophobic polymers including acrylic resins, waxes, higher aliphatic alcohols, polylactic and polyglycolic acids and certain cellulose derivatives such as hydroxypropylmethyl cellulose. In addition, controlled release may be effected by using other polymer matrices, liposomes or microspheres.

The agonists of C3aR function may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

For any compound used in the method of the invention, the therapeutically effective dose can be estimated initially from cell culture assays. For example, a dose can be formulated in animal models to achieve a circulating concentration range that includes the IC₅₀ as determined in cell culture (e.g., the concentration of a test agent, which achieves a half-maximal agonism in activity of C3aR or a component in the complement system associated with C3aR function). Such information can be used to more accurately determine useful doses in humans.

Toxicity and therapeutic efficacy of such drugs can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit large therapeutic indices may be employed. The data obtained from these cell culture assays and animal studies can be used in formulating a range of dosage for use in human. In some embodiments the dosage of such compounds lies within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See for example Fingl et al., 1975, in “The Pharmacological Basis of Therapeutics”, Ch. 1 p1).

Dosage amount and interval may be adjusted individually to provide plasma levels of the active agent which are sufficient to maintain C3aR function agonistic or activation effects. Usual patient dosages for systemic administration range from 1-2000 mg/day, commonly from 1-250 mg/day, and typically from 10-150 mg/day. Stated in terms of patient body weight, usual dosages range from 0.02-25 mg/kg/day, commonly from 0.02-3 mg/kg/day, typically from 0.2-1.5 mg/kg/day. Stated in terms of patient body surface areas, usual dosages range from 0.5-1200 mg/m2/day, commonly from 0.5-150 mg/m2/day, typically from 5-100 mg/m2/day.

Alternately, one may administer the compound in a local rather than systemic manner, for example, via injection of the compound directly into a tissue, which may be subcutaneous or omental tissue, often in a depot or sustained release formulation.

Furthermore, one may administer the drug in a targeted drug delivery system, for example, in a liposome coated with tissue-specific antibody. The liposomes will be targeted to and taken up selectively by the tissue.

In cases of local administration or selective uptake, the effective local concentration of the agent may not be related to plasma concentration.

In order that the invention may be readily understood and put into practical effect, particular preferred embodiments will now be described by way of the following non-limiting examples.

### EXAMPLES

### Example 1

### Mouse Gut Ischemia Repefursion (I/R) Injury

### Materials and Methods

Animals

Male C57BL/6 mice, and C3a receptor (C3aR) knockout mice weighing 20-25 grams were used in these studies.

Intestinal I/R Injury Model

Male C57BL/6 or C3aR knockout mice were fasted for around 12 hours before experimentation. Mice were anesthetized with 2% isoflurane and oxygen at 2 L/minute throughout the experiment. Through a midline laparotomy incision, the superior mesenteric artery (SMA) was exposed and temporarily interrupted by placing a tie with a 6-0 nylon suture around the artery. An immediate check of complete shut-off of the blood supply to the small intestine was confirmed visually (ischemia). The abdomen was then covered with moist gauze. After thirty minutes of the ischemia episode, the ligature was removed and restoration of blood supply to the intestine was confirmed visually (reperfusion). The midline laparotomy incision was then closed. Mice were administrated with saline (100 μL) intraperitoneally every hour to compensate for fluid loss. The same procedure was performed on sham-operated mice including a loop of ligature around their arteries; however the arteries were not occluded. After two and half hours of the reperfusion episode, mice were sacrificed, and tissues and blood were harvested. Their small intestines (0.5-1 cm pieces) were snap-frozen in liquid nitrogen and stored at −80° C. for later assays. Whole blood was collected through the inferior vena cava in a syringe containing 2.5 μL of 200 mg/ml EDTA and 10 μL of 5 mg/mL FUT-175 (for 5004 of blood). The blood was centrifuge at 14,000 rpm for 15 min at 4° C., and the resulting plasma stored at −80° C.

Drug Administration

The C3aR peptide agonist, WWGKKYRASKLGLAR [SEQ ID NO:3] (EP141) (Ember et al., 1991), was administered at 0.01 mg/kg (i.v. in 100 μL saline) to two additional groups of mice (C57BL/6 wild type and C57BL/6 C3aR^{-/-}) 10 minutes prior to the induction of ischemia as described above. A vehicle control group (i.v. 100 saline) was included for direct comparison.

Differential Stain (White Blood Cell Differentials)

To determine white blood cell populations, cell differentials were employed using blood smears and The Kwik-Diff Staining System (Thermo Shandon). At sacrifice, a film of blood obtained from tail blood on a microscope slide was prepared and allowed to dry. The slide was dipped into methanol (Solution #1) for fifteen minutes to fix and permeabilize cells. The slide was then dipped into Xanthene Dye (Solution #2) for three minutes to stain cytoplasm. Next the slide was dipped into Thiazine Dye (Solution #3) for three minutes to stain nuclei. After staining, the slide was rinsed and allowed to air dry (warm air) before mounting. Samples were counted in triplicate, with a total 100 white blood cells per slide counted, and the number of neutrophils expressed as a percentage of total white blood cells.

Myeloperoxidase (MPO) Level Measurement in Small Intestine

Snap-frozen ileum (1 cm), excised at sacrifice, was briefly cleaned with ice-cold PBS and weighed. The tissue was homogenized for 20 seconds in ice-cold 10 mM PBS (pH 6.0) containing 1% protease inhibitor and 0.5% hexadecyltrimethylammonium (HTAB) and sonicated for 10 seconds. After centrifugation (14,000 rpm) for 15 min, the resulting supernatant was assayed. MPO activity was assayed spectrophotometrically at the absorbance 460 nm. The supernatant was diluted in 10 mM PBS (pH 6.0) and mixed with substrate solution containing 2.85 mg/mL o-dianisidine and 0.075% hydrogen peroxide 30%. The absorbance was measured using a Rainbow 96-well plate reader after 20 minutes.

Alkaline Phosphatase (ALP) Measurement

Intestinal ALP concentrations were measured according to the manufacturer's instructions (ALP reagents; Thermo Scientific, USA). Snap-freezing ileum (0.5 cm) was homogenized in ice-cold PBS, and centrifuged at 14,000 rpm at 4° C. for 15 minutes. The supernatant was mixed and incubated with Reagent 1 for 5 minutes at 37° C., and then incubated with Reagent 2. The sample and reagent ratios are 1:40 with Reagent 1 and 1:10 with Reagent 2. The absorbance at 405 was immediately measured in a kinetic manner for 10 minutes at 37° C. The results were calculated as follows: Activity in U/L=ΔAbs/min×Factor.

Histopathology

At sacrifice, a section of ileum (0.5 cm) was removed and fixed in 4% paraformaldehyde in phosphate buffered saline (PBS) for at least 72 hours. The tissue was processed through an increasing series of alcohol dilutions and embedded in paraffin wax. 6 μm sections were cut and stained with hematoxylin-eosin. Histological assessment was carried out in a blinded fashion using light microscopy at 40× magnification, and scored for severity on a scale of 0-5. The grading scheme for intestinal injury was adapted from the method of Chiu et al. 1970.

Cytokines

Cytokines were measured using a Cytometric Bead Array from Becton Dickinson (flow cytometry that allows for the quantification of multiple cytokines simultaneously), using plasma isolated from mice at termination.

### Results

Neutrophil Mobilization

The effect of the absence of C3aR, and agonism of C3aR, to neutrophil mobilization in I/R induced intestinal injury is shown in FIG. 1, where the left panel shows that the percentage of circulating neutrophils was significantly (P<0.0001) higher in C3aR KO mice than wild-type (WT) mice following I/R injury. The same results are indicated in the left two bars of FIG. 2. The right panel shows that the percentage of circulating neutrophils was significantly (P<0.02) lower in C3aR agonist (EP 141) pre-treated WT mice compared with vehicle pre-treated WT mice. The data are expressed as mean±SEM; n=5-9 per group.

FIG. 2 shows the effects of EP141 on circulating neutrophils in both the wild type mice and the C3aR^{-/-} mice in this gut ischemia/reperfitsion model. As can be seen in FIG. 2, EP141 is inactive when administered to the C3aR knockout mice, which demonstrates that EP141 is acting specifically through the C3a receptor in order to reduce neutrophil mobilization.

Neutrophil Activity

The effect of the absence of C3aR, and the agonism of C3aR, on neutrophil activity following I/R induced intestinal injury is shown in FIG. 3, where myeloperoxidase (MPO) activity of the small intestine is shown. On the left panel, it can be seen that the I/R induced intestinal MPO activity was significantly (P<0.01) greater in C3aR KO mice, compared with wild-type (WT) mice. On the right panel, it can be seen that the I/R induced MPO activity was significantly (P<0.05) lower in C3aR agonist (EP141) pre-treated WT mice, compared with vehicle pre-treated WT mice. The data are expressed as mean±SEM; n=10-17 per group.

Intestinal ALP

The effect of the absence of C3aR, and agonism of C3aR, on plasma Alkaline Phosphatase (ALP) following I/R induced intestinal injury is shown in FIG. 4. As shown in the left panel, plasma ALP levels were higher (P=0.057) in C3aR KO mice compared to wild-type (WT) mice. As shown in the right panel, plasma ALP levels were significantly (P<0.02) reduced by pre-treatment with the C3aR agonist (EP141) in WT mice, compared with vehicle pre-treated WT mice. Data are expressed as mean±SEM; n=6-17 per group.

Induced Intestinal Injury

The effect of the absence of C3aR, and agonism of C3aR, to intestinal I/R induced mucosal injury in the mouse small intestine is shown in FIGS. 5A and 5B. The results are shown as histopathological scores of the small intestine. In FIG. 5A, it can be seen that I/R induced intestinal injury was significantly enhanced in C3aR KO mice, compared with wild-type (WT) mice. FIG. 5B shows that I/R induced intestinal injury was significantly reduced in C3aR agonist (EP141) pre-treated WT mice compared with vehicle pre-treated WT mice. The data are expressed as mean±SEM. *p50.05 and ***p50.001; n=9-15 per group.

The results in FIG. 5 are different graphs to those originally provided (color bar graphs).

Cytokines

Levels of IL-113, TNF-α, IL-6, IL-10 and IL-23 were measured in the plasma of the wild type sham mice, wild type ischemia/reperfusion mice, the C3aR^{-/-} isclemia/reperfusion mice and the wild type ischemia/reperfusion mice who had received. EP141, 2.5 hours after ischemia (or after sham surgery). Only IL-6 was detectable, and therefore only this data is presented. FIG. 6 shows the IL-6 levels measured in each group, demonstrating that there was no significant change in the IL-6 levels in the C3aR^{-/-} mice and the wild type mice treated with EP141.

### Discussion

In the experiments described in Example 1, the role of C3a was characterized using both wild-type C57BL6/J mice or C57BL6/J mice deficient in C3a receptors (C3aR^{-/-}). Pathology outcomes of the mice were examined following gut ischemia-reperfusion. The C3aR^{-/-} mice displayed exacerbated pathology following disease onset, compared to the wild-type mice. These results suggest that C3aR activation reduces the inflammatory response.

To further explore the anti-inflammatory potential of the C3aR, a C3aR agonist was administered to wild-type mice prior to gut ischemia-reperfusion, and was found to significantly ameliorate inflammatory disease. A protective effect was observed in mice administered the C3aR agonist, despite these mice already having endogenous protection provided through endogenous C3a generation and interaction with C3aR. These studies indicated that C3aR is an anti-inflammatory receptor in this experimental model, and that pharmacological targeting of C3aR can counter inflammation in experimental model of the disease, above and beyond what is already provided for by C3a endogenously.

Furthermore, the C3aR agonist (EP141) was shown to be acting specifically through the C3a receptor in order to reduce neutrophil mobilization

The fact that circulating cytokine levels, with the exception of IL-6, were unchanged in this acute model, suggests that the majority of pathology occurring in this model is not through inflammatory cytokine generation. In the case of the elevation of IL-6, despite C3aR^{-/-} mice, and EP141 therapy, providing substantial changes to the disease outcome in these mice, this cytokine was not affected by any treatment. This further suggests that the mechanism of protection of C3aR activation in this model, is not due to a change in soluble cytokine production.

### Example 2

### Spinal Cord Injury (SCI)

### Materials and Methods

Experimental Spinal Cord Injury

Experimental mice were anaesthetized using Zoletil (50 mg/kg) and xylazine (10 mg/kg). Next, a dorsal laminectomy was performed on the 9^{th} thoracic vertebra to expose the underlying spinal cord. Mice were then subjected to a force-controlled, moderate-severe (70 kdyne) contusion injury of the spinal cord using the Infinite Horizon impactor device (Ghasemlou et al., 2005). Post-operatively, mice were given antibiotics for 5 days (Gentamycin; 1 mg/kg) as well as a single dose of Hartmann's solution (1 ml) to prevent dehydration and to compensate for loss of bodily fluids. Bladders of spinal cord-injured mice were voided manually twice daily. C3aR^{-/-} and wild-type mice were sacrificed at 35-42 days post-injury (n=16 per experimental condition and time point); these time points, were specifically chosen because they coincide with the point in time where no further improvements are normally seen in spontaneous recovery of function (day 42), see Basso et al., 2006 and Kigerl et al., 2006.

Functional Assessment

To assess the impact of C3aR deficiency on the functional outcome from SCI, locomotor performance of spinal cord-injured C3aR^{-/-} and wild-type mice was assessed in open field at 1, 3, 5 and 7 days post-injury plus weekly thereafter up to the late survival time point of 42 days post-injury. Experimental mice received neurological scores based on the Basso Mouse Scale (BMS); this scoring method was specifically developed to assess functional deficits in mice with contusive SCI (Basso et al., 2006). Of note, BMS scores were assigned to spinal cord-injured mice by two independent investigators who were at all times blinded to the experimental conditions.

Magnetic Resonance Imaging (MRI)

Non-invasive MRI was used to assess the impact of C3aR deficiency on SCI-associated neuropathology at a gross anatomical level.

For post-mortem MRI, mice with contusive SCI were sacrificed at the specified survival time points of 1, 3, 7, 14 and 42 days post-injury via perfusion fixation with Zamboni's fixative solution. Following dissection of the vertebral column, high-resolution MRI was used to compare tissue integrity and core lesion size between experimental groups.

For quantitative assessment of lesion size, samples were incubated for 24-48 hrs with contrast agent (0.2% Magnevist), followed by imaging on a 16.4-Tesla small animal MRI (UQ Centre for Advanced Imaging; CID Cowin). Imaging time per sample was approximately 2.5 hrs, yielding an isotropic image resolution of 32 microns. Avizo® software was used for masked image analysis and accurate 3D reconstruction and quantification of the lesion size.

Assessment of Inflammation and Neuropathology after SCI

To further investigate the role of C3aR at a cell and tissue level, routine histological procedures and flow cytometry were employed as follows.

For histological examination, following conclusion of post-mortem MRI, C3aR^{-/-} and wild-type spinal cords were dissected from the vertebral column, cryo-protected and snap-frozen in cold iso-pentane. Coronal spinal cord sections (16 μm) were cut on a cryostat and collected on slides. Antibody staining was used to compare and contrast histopathological features of the injury site between time points and genotypes:

(1) White matter sparing and astrogliosis at the lesion epicenter was assessed via staining with FluoroMyelin and antibodies again the astrocyte marker ‘glial acidic fibrillary protein’ (GFAP), respectively. Proportional area measurements, i.e., stained area relative to the total section area, were used as a quantitative measure to compare genotypes. Double staining for C3aR was used to determine which of these cells co-express this complement anaphylatoxin receptor; and

(2) Neutrophil infiltration at the lesion site was characterized via immunostaining for Ly6G and/or neutrophil-specific clone “7/4”. Unbiased stereological counts or ‘proportional area’ measurements were used to compare outcomes between experimental time points and genotypes (Kigerl et al., 2006 and Ruitenberg et al., 2004).

Generation of BM Chimeric Mice

To specifically assess the effect of conditional C3aR deficiency in the bone marrow (BM) and on circulating leukocytes for the outcome from SCI, [C3aR^{-/-}>WT] BM chimeric mice were created. In brief, recipient mice were sub-lethally irradiated (2×5Gy; 14 hrs apart) to deplete the hematopoietic stem cell niche, followed by BM transplantation. In brief, donor BM was harvested from the femur and tibia, subjected to red blood cell lysis and administered via injection into the lateral tail vein (2-3 hours after the second dose of irradiation; 5×10⁶ cells in 150 μl), see Vukovic et al., 2010 and Chinnery et al., 2010. BM chimeric mice were let to recover for 8 weeks before being subjected to SCI.

The validity and efficiency of the chimerization process was tested in congenic [CD45.1>CD45.2] BM chimeras (CD45 being a marker for all nucleated BM-derived cells) and subsequent flow cytometric analysis of blood samples. Isoform-specific antibodies revealed that 93±0.01% of circulating cells were of donor BM origin at 8 weeks post-chimerization (n=10 mice).

BM Chimeric Mice and SCI

Following an 8-week recovery period, BM chimeric mice were subjected to SCI as detailed above. Functional recovery was monitored again via open-field locomotor scoring as detailed above.

Blood Sample Collection and Analysis

Blood samples were collected from experimental animals either via retro-orbital eye bleeds or heart punctures at the time of sacrifice. Part of the blood was used for flow cytometry to assess the relative populations of white blood cells in normal and SCI mice as well as in response to treatment with C3aR agonist, EP67 (see below). The remainder of the sample was used to compare ‘total white blood cell (WBC) counts’ between wild-type and C3aR^{-/-} mice under homeostatic and SCI conditions. For ‘total WBC counts’, blood samples were subjected to red blood cell lysis using routine procedures (ammonium chloride). Next, the number of white blood cells per set sample volume was determined using the Countess automated cell counter (Invitrogen) as per manufacturer's instructions. These experiments were designed to reveal the impact of spinal cord injury and/or C3aR deficiency on leukocyte numbers in the circulation as a function of post-injury time.

Pharmacological Activation of C3aR Following SCI

These experiments were performed to test whether C3aR can be used as a therapeutic target to improve the outcome from neurotraumatic events (neuronal death and axonal degeneration at sites of SCI are inevitably linked to neuroinflammation). The C3aR agonist, EP67, was administered during the acute phase of SCI to probe the use of this complement receptor as a therapeutic target. Experimental mice with SCI received four consecutive dosages of EP67 (1 mg/kg s.c.), with the first dose administered immediately after injury and repeat dosages every 6 hours thereafter or saline as a control. Mice were sacrificed at 24 hours post-injury. The effect of EP67 treatment on circulating white blood cell populations after SCI relative to saline-treated controls was assessed via flow cytometry and total WBC counts as detailed above.

### Results

C3aR Deficiency Worsens the Outcome from SCI

As shown in FIG. 7, mice that are deficient in C3aR, i.e., lacking the G protein-coupled receptor via which C3a exerts its effects on the inflammatory response, have a significantly worsened outcome from SCI (i.e., lower BMS scores) with decreased locomotor performance, increased lesion volumes, reduced white matter sparing and increased neutrophil presence at 35 days post-injury, compared to wild-type mice. See, especially, FIG. 7A (n=10-12 per group). Of note, a score ‘9’ represents normal locomotion while ‘0’ is total paralysis (Basso et al., 2006). In accord with the functional data, lesion volumes were larger in C3aR^{-/-} mice as determined by MRI, see the arrows in FIG. 7B, and the subsequent quantitative analysis in FIG. 7C. The amount of myelin in spared white matter was also significantly decreased as shown in FIG. 7D (the light grey is the white matter) and shown quantitatively in FIG. 7E. Finally, increased neutrophil presence was evident at the lesion site in C3aR^{-/-} mice as shown in FIG. 7F and FIG. 7G (n≧6; *, p<0.05). These results suggest that C3a serves a reparative role following neurotraumatic events.

C3aR is expressed by neutrophils, astrocytes and macrophages in the injured spinal cord.

The results also show that C3aR itself is expressed in the injured spinal cord and its expression is localized to neutrophils, astrocytes and activated microglia/macrophages at the lesion site for at least up to 35 days post-injury. These results are shown in FIG. 8 where the images shows differential staining for C3aR (left image), GFAP⁺ astrocytes and Iba-1⁺ macrophages (both in middle image), and merged image (right image). The staining shows that C3aR is present on GFAP⁺ astrocytes and Iba-1⁺ macrophages after SCI, and that C3aR⁺ neutrophils do not co-express these markers (arrowhead).

Expression of C3aR on BM-derived cells is critical for the outcome from SCI.

By transplanting C3aR-deficient BM into wild-type recipient mice, i.e. [C3aR^{-/-}>WT] BM chimeras, this experiment also tested the importance of C3aR expression in the BM and on circulating leukocytes for the outcome from SCI. The data indicate that the functional phenotype of C3aR deficiency is not rescued in these BM chimeras where C3aR expression is restricted to CNS astrocytes and microglia, as shown in FIG. 9. In FIG. 9, it can be seen that the [C3aR^{-/-}>WT] BM chimeric mice have a worse, functional outcome from SCI compared to the injured controls. Thus, expression of C3aR on astrocytes and microglia in [C3aR^{-/-}>WT] BM chimeric mice does not rescue the phenotype observed in C3aR-deficient mice. Transplantation of wild-type (WT) bone marrow into irradiated WT recipients was used to exclude the possibility that the chimerization process influenced the outcome from SCI (n=10 per condition; *, p<0.05 [ANOVA & post-hoc]). The presence of C3aR on BM-derived cells is thus crucial for the outcome from SCI.

EP67 treatment reduces circulating granulocyte numbers after SCI.

The comparison of total WBC count revealed no difference between wild-type and C3aR-deficient mice under homeostatic conditions (data not shown). The relative distribution of the major white blood cell populations (i.e., lymphocytes, monocytes and granulocytes) was also normal and not different between genotypes. It was, however, noted that SCI causes a dramatic increase in the number of circulating granulocytes at 24 hours post-injury (see FIG. 10, left hand graph as discussed below). Further, treatment with EP67 countered the granulocyte egress from the bone marrow, resulting in a 30% reduction in the blood granulocyte number compared to vehicle-treated control mice with SCI (see, FIG. 10, right hand graph, discussed below). A trend towards a decrease in the total WBC count was also observed following EP67-treatment relative to vehicle-treated controls with SCI (data not shown; p=0.07).

FIG. 10 shows two graphs. In FIG. 10, the bars represent mean±SEM; n=4 per experimental condition; asterisk indicates p<0.05.

The left hand graph in FIG. 10 shows the white blood cell distributions (first two bars=lymphocytes; second pair of bars=granulocytes; third pair=monocytes) in uninjured controls (the left bars of each pair—“baseline”) and mice with experimental SCI (the right bars of each pair) at 24 hours post-injury. A dramatic increase in granulocyte numbers in SCI mice as a result of their mobilization in response to injury is shown.

The right hand graph in FIG. 10 shows the white blood cell distributions (first two bars=lymphocytes; second pair of bars=granulocytes; third pair=monocytes) 24 hours after SCI, where the left bars of each pair show the results for the saline control and the right bars of each pair show the results for treatment of experimental mice with the C3aR agonist EP67. It can be seen that the EP67 treatment partially counters the granulocyte egress from the BM in response to SCI, resulting in a significant 30% reduction in blood granulocyte numbers.

### Discussion

Deficiency in C3aR worsened the outcome from SCI. Based on the data from C3aR-deficient mice with SCI, it is predicted that C3aR agonism during the acute phase of SCI would be anti-inflammatory and thus prove beneficial in relation to the outcome from SCI. These findings indeed confirm C3aR as a putative therapeutic target in SCI.

### Example 3

### Screening Assay for C3a Receptor (C3aR) Agonists

As shown in the aforementioned Examples, following IR injury, mice deficient in C3aR (C3aR^{-/-}) had significantly more circulating neutrophils than WT mice and administration of a C3aR agonist to WT mice substantially reduced neutrophil activity and the number of circulating neutrophils. Thus, there is evidence that C3aR is an anti-inflammatory receptor and that pharmacological targeting of C3aR can counter inflammation, above and beyond what is already provided for by C3a endogenously. The present invention includes a method of screening for C3aR agonists.

Cells, e.g., rat basophilic leukemia cell line, RBL-2H3 (ATCC no. CRL-2256) stably expressing human C3aR (polynucleotide as set forth for example in GenBank Accession No. NM_{—}004054; polypeptide, as set forth for example in GenPept Accession No. NP_{—}004045) are maintained under standard cell culture conditions in Eagle's MEM with Earle's salts, with L-glutamine and nonessential amino acids supplemented with FBS (10%) and G418 (400 μg/mL). A radioligand binding assay, based on the use of C3aR-expressing cells, or membranes of cells thereof, and ¹²⁵I-labelled human C3a (polynucleotide comprising for example the nucleotide sequence: tccgtgcagctcacggagaagcgaatggacaaagtcggcaagtaccccaaggagctgcgcaagtgctgcgaggacggcatg cgggagaaccccatgaggttctcgtgccagcgccggacccgtttcatctccctgggcgaggcgtgcaagaaggtcttcctgga ctgctgcaactacatcacagagctgcggcggcagcacgcgcgggccagccacctgggcctggccagg [SEQ ID NO:16]; polypeptide comprising for example the amino acid sequence: SVQLTEKRMDKVGKYPKELRKCCEDGMRENPMRFSCQRRTRFISLGEACKKVFL DCCNYITELRRQHARASHLGLAR [SEQ ID NO:1]) is performed in a 96-well microtiter plate format. Live cells, or membranes thereof, are bound to beads. Each plate well contains ¹²⁵I-labelled C3a in binding buffer. Control wells, used to measure non-specific binding, include an excess of unlabelled C3a. Putative modulators of C3aR, such as non-peptide (e.g., small molecules from chemical libraries), or peptide, compounds are added. After incubation, the plates are washed and the plate-bound radioactivity is counted on a scintillation counter. The greater the binding of a test compound to the receptor (indicative of possible agonist or antagonist activity), the less of the radiolabeled ligand will bind to the cells or the cell membranes bound to the plate, which in turn results in a lower radioactivity count.

Compounds with high affinity binding to the respective receptor are further tested for an agonistic effect in another assay to distinguish agonists from antagonists, e.g., in a receptor internalization assay, a calcium mobilization assay, a H₂O₂ production assay, a cell proliferation assay, a cell differentiation assay, and/or in an animal model of stroke or neural injury, etc. Exemplary assays are described herein.

In one variation, a receptor internalization assay uses human neutrophils which are stimulated by incubation with the natural ligand C3a or with putative agonist compounds. C3aR are detected by polyclonal antibodies, and receptor internalization is quantified by flow cytometry. Increased receptor internalization correlates with reduced levels of receptors detected on the cell surface. Compounds that induce an effect similar to, or greater than, the natural ligand are regarded as agonists.

A calcium mobilization assay uses human neutrophils which are incubated with a calcium fluorescent indicator (e.g., Fura-2, Fluo-4) for 60 min at 37° C. After a washing step, neutrophils (5×10⁶ cells/mL) are loaded into a 96-well black welled plated and different concentrations of C3a polypeptide or putative agonist compounds administered. The fluorescent change (mobilization of calcium in response to C3aR activation) is measured in an appropriate machine (eg. Flexstation 3 or FLIPR).

A H₂O₂ production assay determines H₂O₂ production by isolated human neutrophils. In this assay, cells are pre-treated for 1 h at 37° C. with C3a polypeptide (@ 10 nM) or a putative agonist compound(s) in the presence of 1 mM sodium azide (to prevent endogenous catalases from destroying H₂O₂). To stimulate neutrophils, phorbol myristate acetate (25 ng/mL) is added at the end of the pre-incubation and cell suspensions are incubated at 37° C. for 15 min. The reaction is terminated by the addition of 0.1 mL of trichloroacetic acid (50% w/v). Samples are then centrifuged for 10 mM at 500×g, and ferrous ammonium sulfate and potassium thiocyanate are added to the supernatant at final concentrations of 1 S mM and 0.25 M, respectively. The absorbance of the ferrithiocyanate complex is measured at 480 nm and is compared with a standard curve generated from dilutions of reference solutions of H₂O₂.

A compound identified as a putative C3aR agonist is also tested for its effect(s) on neutrophil mobilization, tissue infiltration and activity as illustrated for example in Examples 1 and 2.

C3a ligands, fragments thereof, and the C3aR agonist compounds, identified by the methods of the present invention, are then used to treat or prevent an acute inflammatory condition, including ones that are characterized by increased neutrophil mobilization and/or increased neutrophil infiltration of tissue and/or increased neutrophil activity, as compared for example to normal subjects or subjects without the acute inflammatory condition.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

Basso D M, Fisher L C, Anderson A J, Jakeman L B, McTigue D M, Popovich P G “Basso Mouse Scale for locomotion detects differences in recovery after spinal cord injury in five common mouse strains” 2006 J Neurotrauma 23: 635-59.
Button D and Brownstein M, “Aequorin-expressing mammalian cell lines used to report Ca²⁺ mobilization” 1993 Cell Calcium 14: 663-671.
Chinnery H R, Ruitenberg M J, McMenamin P G “Novel characterisation of monocyte-derived cell populations in the meninges and choroid plexus and their rates of replenishment in bone marrow chimeric mice” J Neuropathol Exp Neurol 69: 896-909.
Chiu C J, McCardlea H, Brown R, Scott H J, Gurdf N. “Intestinal mucosal lesion in low flow states” 1970 Arch Surg 101: 478-483.
Denonne F, Binet S, Burton M, Collart P, Dipesa A, Ganguly T, Giannaras A, Kumar S, Lewis T, Maounis F, Nicolas J-M, Mansleu T, Pasau P, Preda D, Stebbins K, Volosov A, Zou D, “Discovery of new C3aR ligands. Part 1: Arginine derivatives” 2007 Bioorganic & Medicinal Chemistry Letters 17: 3258-3261.
Denonne F, Binet S, Burton M, Collart P, Defays S, Dipesa A, Eckert M, Giannaras A, Kumar S, Levine B, Nicolas J-M, Pasau P, Pegurier C, Preda D, Van houtvin N, Volosov A, Zou D, “Discovery of new C3aR ligands. Part 2: Amino-piperidine derivatives” 2007 Bioorganic & Medicinal Chemistry Letters 17: 3262-3265.
Ember J A, Johansen N L, Hugli T E, “Designing synthetic superagonists of C3a anaphylatoxin” Apr. 16, 1991 Biochemistry 30(15): 3603-3612. Erratum in Jun. 25, 1991 Biochemistry 30(25): 6350.
Fukuoka Y, Yasui A, Tachibana T, “Active recombinant C3a of human anaphylatoxin produced in Escherichia coli” 1991 Biochem Biophys Res Commun 175(3): 1131-1138.
Gerardy-Schahn R, Ambrosius D, Casaretto M, Grotzinger J, Saunders D, Wollmer A, Brandenburg D, Bitter-Suermann D, “Design and biological activity of a new generation of synthetic C3a analogues by combination of peptidic and non-peptidic elements” 1988 Biochem J 255: 209-210.
Ghasemlou N, Kerr B J, David S “Tissue displacement and impact force are important contributors to outcome after spinal cord contusion injury” 2005 Exp Neurol 196: 9-17.
Kigerl K A, McGaughy V M, Popovich P G “Comparative analysis of lesion development and intraspinal inflammation in four strains of mice following spinal contusion injury” 2006 J Comp Neurol 494: 578-94.
Klos A, Ihrig V, Messner M, Grabbe J, Bitter-Suermann D, “Detection of native human complement components C3 and C5 and their primary activation peptides C3a and C5a (anaphylatoxic peptides) by ELISAs with monoclonal antibodies” 1988 J Immunological Methods 111(2): 241-252.
Li J, Pircher P C, Schulman I G, Westin S K, “Regulation of Complement C3 Expression by the Bile Acid Receptor FXR” 2005 JBC 280(9): 7427-7434.
Mathieu M-C, Sawyer N, Greig G M, Hamel M, Kargman S, Ducharme Y, Lau C K, Friesen R W, O'Neill G P, Gervais F G, Therien A G, “The C3a receptor antagonist S B 290157 has agonist activity” Sep. 15, 2005 Immunology letters 100(2): 139-145.
Mottheakis L C and Ohler L D, “Seeing the light: calcium imaging in cells for drug discovery” 2000 Drug Discovery Today: HTS Supplement 1(1): 18-19.
Rizutto R, Brini M, Bastianutto C, Marsault R, Pozzan T, “Photoprotein-mediated measurement of calcium ion concentration in mitochondria of living cells” 1995 Methods in Enzymology 260: 417-428.
Ruitenberg M J, Blits B, Oudega M, teBeek E T, Bakker A, van Heerikhuize J J, Pool C W, Hermens W T, Boer G J, Verhaagen J “Adeno-associated viral vector-mediated gene transfer of brain-derived neurotrophic factor reverses atrophy of rubrospinal neurons following both acute and chronic spinal cord injury” 2004 Neurobiol Dis 15: 394-406.
Scully C C G, Blakeney J S, Singh R, Hoang H N, Abbenante G, Reid R C, Fairlie D P, “Selective Hexapeptide Agonists and Antagonists for Human Complement C3a Receptor” 2010 J. Med. Chem. 53(13): 4938-4948.
Segel G B, Halterman M W, Lichtman M A, “The paradox of the neutrophil's role in tissue injury” March 2011 J. Leukoc. Biol. 89: 359-372.
Sheu Y-A, Kricka L J, Pritchett D B, “Measurement of intracellular calcium using bioluminescent aequorin expressed in human cells” 1993 Analytical Biochemistry 209: 343-347.
Takenaka Y, Utsumi S, Yoshikawa M, “Introduction of a low molecular weight agonist peptide for complement C3a receptor into soybean proglycinin A1aB1b subunit by site-directed mutagenesis” May 2001 Biosci Biotechnol Biochem 65(5): 1202-1205.
Vogen S M, Paczkowski N J, Kimarsky L, Short A, Whitmore J B, Sherman S A, Taylor S M, Sanderson S D, “Differential activities of decapeptide agonists of human C5a: the conformational effects of backbone N-methylation” November 2001 Int Immunopharmacol. 1(12): 2151-2162.
Vukovic J, Blomster L V; Chinnery H R, Weninger W, Jung S, McMenamin P G, Ruitenberg M J “Bone marrow chimeric mice reveal a role for CX3CR1 in maintenance of the monocyte-derived population in the olfactory epithelium” 2010 J Leukoc Biol 88: 645-54.
Woodruff T M, Arumugam T V, Shiels I A, Reid R C, Fairlie D P, Taylor S M, “Protective effects of a potent C5a receptor antagonist on experimental acute limb ischemia-reperfusion in rats” January 2004 J. Surg. Res. 116(1): 81-90.
Woodruff T M, Arumugam T V, Shiels I A, Reid R C, Fairlie D P, Taylor S M, “A potent human C5a receptor antagonist protects against disease pathology in a rat model of inflammatory bowel disease” 15 Nov. 2003 J Immunol. 171(10): 5514-5520.

1. (canceled)

2. (canceled)

3. A method for preventing or treating an acute inflammatory condition in an individual, the method comprising administering to the individual an effective amount of an agonist of C3aR function.

4. The method according to claim 1, wherein the acute inflammatory condition is characterized by any one or more of increased neutrophil mobilization, increased neutrophil infiltration of tissue or increased neutrophil activity.

5. The method according to claim 1, wherein the acute inflammatory condition is characterized by increased neutrophil mobilization.

6. The method according to claim 1, wherein the agonist of C3aR function has undergone testing to determine that it agonizes C3aR function.

7. The method according to claim 6, wherein the testing comprises determining that the agonist binds to C3aR.

8. The method according to claim 6, wherein the testing comprises determining that the agonist induces C3aR activation.

9. The method according to claim 6, wherein the testing comprises determining that the agonist induces C3aR internalization.

10. The method according to claim 6, wherein the testing comprises determining that the agonist induces C3aR signaling.

11. The method according to claim 6, wherein the testing comprises determining that the agonist induces intracellular calcium mobilization.

12. The method according to claim 6, wherein the testing comprises determining that the agonist induces granular enzyme release.

13. The method according to claim 6, wherein the testing comprises determining that the agonist reduces neutrophil mobilization.

14. The method according to claim 6, wherein the testing comprises determining that the agonist reduces neutrophil infiltration of tissue.

15. The method according to claim 6, wherein the testing comprises determining that the agonist reduces the level or activity of neutrophils.

16. The method according to claim 6, wherein the testing comprises determining that the agonist reduces inflammation.

17. The method according to claim 6, wherein the acute inflammatory condition is selected from the group consisting of: ischemia, ischemia-reperfusion injuries, hemorrhagic shock, conditions associated with trauma, transfusion related acute lung injury (TRALI), acute respiratory distress syndrome (ARDS), trauma or injury associated with surgery, renal microvasculopathy, acute renal failure, vasculitis, pancreatitis, dermatitis, acute coronary artery syndrome, acute liver injury, acute bacterial infection, and acute tissue injury.

18. The method according to claim 6, wherein the agonist of C3aR function is a peptide or polypeptide.

19. The method according to claim 18, wherein the agonist of C3aR function is a peptide that comprises, consists or consists essentially of the naturally-occurring human C3a sequence or an amino acid sequence corresponding to naturally-occurring human C3a sequence.

20. The method according to claim 18, wherein the agonist of C3aR function is a peptide that comprises, consists or consists essentially of the C-terminal region of the naturally-occurring human C3a sequence, suitably residues 63-77, or an amino acid sequence corresponding to that C-terminal region.

21. The method according to claim 20, wherein the agonist of C3aR function is a peptide that comprises, consists or consists essentially of WWGKKYRASKLGLAR [SEQ ID NO:3]

22. The method according to claim 18, wherein the agonist of C3aR function is a peptide represented by an amino acid sequence having the formula:
X_{N}X₁X₂X₃X₄X₅X_{C}
wherein:
X_{N} is the N-terminus of the peptide comprising a sequence corresponding to at least a fragment of the amino acid sequence defined by residues 1 to 72 of the naturally-occurring human C3a;
X₁ is a hydrophobic amino acid residue or modified form thereof;
X₂ is a small amino acid residue or modified form thereof;
X₃ is leucine or a modified form thereof;
X₄ is a small amino acid residue or modified form thereof;
X₅ is arginine or a modified form thereof; and
X_{C} is the C-terminus of the peptide which comprises from 0 to 5 amino acid residues.

23. The method according to claim 22, wherein the peptide is selected from the group consisting of LPLR [SEQ ID NO:4], PSYLPLPR [SEQ ID NO:5], RPSYLPLPR [SEQ ID NO:6], FLPLAR [SEQ ID NO:7], FIPLAR [SEQ ID NO:8], FWTLAR [SEQ ID NO:9] and FLTLAR [SEQ ID NO:10], HLGLAR [SEQ ID NO:11], HLALAR [SEQ ID NO:12], YSFKDMP(MeL)aR [SEQ ID NO:13] and YSFKPMPLaR [SEQ ID NO:14].

24. The method according to claim 22, wherein the peptide is LPLPR.

25. The method according to claim 6, wherein the agonist of C3aR function is a small organic molecule.

26. The method according to claim 25, wherein the agonist of C3aR function is a biaryl-substituted arginine compound or a triarylmethine-substituted arginine compound.

27. The method according to claim 26, wherein the agonist of C3aR function has the structure: 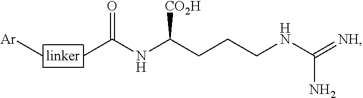
wherein:
the aryl group comprises 3-chlorophenyl, and
the linker comprises 2,5-furyl.

28. The method according to claim 26, wherein the agonist of C3aR function has the structure: 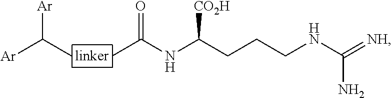
wherein:
the diarylmethine group comprises 3-fluorophenyl, and
the linker comprises 2,5-furyl;

29. The method according to claim 26, wherein the agonist of C3aR function has the structure: 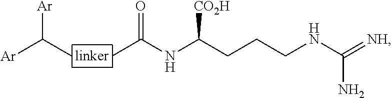
wherein:
the diarylmethine group comprises 3-chlorophenyl, and
the linker comprises 2,5-furyl; and

30. The method according to claim 26, wherein the agonist of C3aR function has the structure: 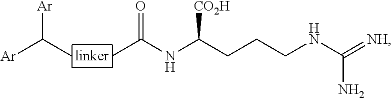
wherein:
the diarylmethine group comprises 4-chlorophenyl, and
the linker comprises 2,5-furyl.

31. The method according to claim 25, wherein the agonist of C3aR function is an amino-piperidine derivative.

32. The method according to claim 31, wherein the agonist of C3aR function has the structure: 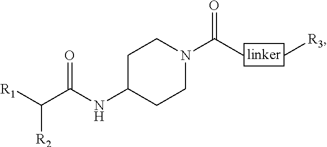
wherein:
R1 comprises phenyl, R2 comprises cyclohexyl, the linker comprises C₂H₄, and R3 comprises 3-pyridyl;
R1 comprises 3-chlorophenyl, R2 comprises cyclohexyl, the linker comprises C₂H₄, and R3 comprises 3-pyridyl;
R1 comprises 3,4,5-trifluorophenyl, R2 comprises cyclohexyl, the linker comprises C₂H₄, and R3 comprises 3-pyridyl;
R1 comprises 2-naphthyl, R2 comprises cyclohexyl, the linker comprises C₂H₄, and R3 comprises 3-pyridyl; or
R1 comprises phenyl, R2 comprises cyclohexyl, the linker comprises C₂H₄, and R3 comprises 2-fluoro-3-pyridyl.

33. The method according to claim 25, wherein the agonist of C3aR function is a benzeneacetamide,α-cyclohexyl-N-[1-[1-oxo-3-(3-pyridinyl)propyl]-4-piperidinyl]-, having the structure:

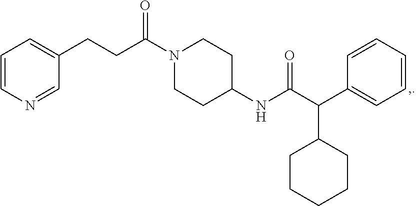 [SMILES: O=C(NC1CCN(C(=O)CCc2cccnc2)CC1)C(c1ccccc1)C1CCCCC1]

34. The method according to claim 25, wherein the agonist of C3aR function is a 5-(diaminomethylideneamino)-2-[[2-(2,2-diphenylethoxy)acetyl]amino]pentanoic acid having the structure:

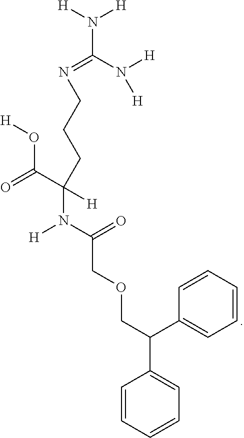 [SMILES: NC(N)=NCCCC(NC(=O)COCC(c1ccccc1)c1ccccc1)C(=O)O]

35. A method of producing a pharmaceutical composition, the method comprising contacting a sample of cells expressing C3aR with a test agent, detecting an increase in level or activity of C3aR relative to a reference or control level in the absence of the test agent, which indicates that the agent agonizes C3aR function, and formulating a pharmaceutical composition comprising the agent for preventing or treating an acute inflammatory condition.

36. The method according to claim 35, further comprising determining whether the agent induces C3aR activation, wherein a detected increase in C3aR activation indicates that the agent agonizes C3aR function and is useful for preventing or treating an acute inflammatory condition.

37. The method according to claim 35, further comprising determining whether the agent induces C3aR internalization, wherein a detected increase in C3aR internalization indicates that the agent agonizes C3aR function and is useful for preventing or treating an acute inflammatory condition.

38. The method according to claim 35, further comprising determining whether the agent induces C3aR signaling, wherein a detected increase in C3aR signaling indicates that the agent agonizes C3aR function and is useful for preventing or treating an acute inflammatory condition.

39. The method according to claim 35, further comprising determining whether the agent induces intracellular calcium mobilization, wherein a detected increase in intracellular calcium mobilization indicates that the agent agonizes C3aR function and is useful for preventing or treating an acute inflammatory condition.

40. The method according to claim 35, further comprising determining whether the agent induces granular enzyme release, wherein a detected increase in granular enzyme release indicates that the agent agonizes C3aR function and is useful for preventing or treating an acute inflammatory condition.

41. The method according to claim 35, further comprising determining whether the agent reduces neutrophil mobilization, wherein a detected decrease in neutrophil mobilization indicates that the agent agonizes C3aR function and is useful for preventing or treating an acute inflammatory condition.

42. The method according to claim 35, further comprising determining whether the agent reduces neutrophil infiltration of tissue, wherein a detected decrease in neutrophil infiltration of tissue indicates that the agent agonizes C3aR function and is useful for preventing or treating an acute inflammatory condition.

43. The method according to claim 35, further comprising determining whether the agent reduces the level or activity of neutrophils, wherein a detected decrease in level or activity of neutrophils indicates that the agent agonizes C3aR function and is useful for preventing or treating an acute inflammatory condition.

44. The method according to claim 35, further comprising determining whether the agent reduces inflammation, wherein a detected decrease in inflammation indicates that the agent agonizes C3aR function and is useful for preventing or treating an acute inflammatory condition.

45. A method of producing an agent comprising an agonist of C3aR function for preventing or treating an acute inflammatory condition in an individual, the method comprising identifying an agent that agonizes C3aR function according to claim 35, and synthesizing the agent on the basis that it tests positive for agonizing C3aR function, optionally further derivatizing the agent, and optionally formulating the derivatised agent with a pharmaceutically acceptable carrier or diluent.
